(19) United States
(12) Patent Application Publication
(10) Pub. No.: US 2024/0382736 A1
(43) Pub. Date: Nov. 21, 2024

(54) System and Method for Vascular Access Management

(71) Applicant: Becton, Dickinson and Company, Franklin Lakes, NJ (US)

(72) Inventors: Yashar Seyed Vahedein, Mountain View, CA (US); Erik Kurt Witt, Oakland, NJ (US); Mark Jesser, Wyckoff, NJ (US)

(21) Appl. No.: 18/694,317

(22) PCT Filed: Sep. 26, 2022
(86) PCT No.: PCT/US22/44667

§ 371 Date: Mar. 21, 2024

Related U.S. Application Data
(60) Provisional application No. 63/248,763, filed on Sep. 27, 2021.

(51) Int. Cl. A61M 39/02; A61M 5/168; G06T 7/70; G16H 40/67

(52) U.S. Cl. CPC A61M 39/0247; A61M 5/16831; G06T 7/70; G16H 40/67; A61M 2039/0258; A61M 2039/0285; A61M 2205/18; A61M 2205/3306; G06T 2207/30004

(57) ABSTRACT

One or more image capture devices may capture, over a period of time, a plurality of images of an environment. One or more processors may determine, based on the plurality of images, a plurality of locations of a plurality of medical devices and a plurality of types of the plurality of medical devices; determine, based on the plurality of locations, a plurality of distances between the plurality of medical devices; determine, based on the plurality of distances and the plurality of types, at least one event of the following events: (i) a connection of a first medical device to a second medical device and (ii) a disconnection of the first medical device from the second medical device; and update, based on the at least one determined event, a database including current connections between the plurality of medical devices.

### CROSS-REFERENCE TO RELATED APPLICATION

The present application claims priority to U.S. Provisional Application Ser. No. 63/248,763, entitled “System and Method for Vascular Access Management”, filed Sep. 27, 2021, the entire disclosure of which is hereby incorporated by reference in its' entirety.

### BACKGROUND

Vascular access management involves many nursing tasks and decisions throughout a patient's stay in care including selecting correct catheters, gathering supplies and preparing catheter insertion sites, inserting catheters, maintaining inserted catheters (e.g., checking insertion sites, changing dressings, changing tubing and connectors, flushing, delivering medications, etc.), and removing catheters. Failures and challenges associated with vascular access management include catheter-related bloodstream infections (CRBSIs), central line associated bloodstream infections (CLABSIs), occlusions, unnecessary vascular device usage, blood vessel damage, catheter dislodgement, phlebitis, medication errors, lack of first stick or insertion success, infiltration/extravasation, treatment delays, and/or the like, which may lead to premature removal of catheters, increased healthcare costs, and lower patient satisfaction.

For example, catheter-associated bloodstream infections are high impact events, with high costs and high mortality. The incidence of catheter-associated bloodstream infections has been estimated at 0.3-0.5% CRBSI for peripheral catheters and 13-20% CLABSI for central lines. The Joint Commission estimates that 250,000 CLABSIs occur each year in the U.S., costing more than $1 billion a year to treat, and resulting in more than 30,000 deaths. Clinicians continue to seek out technologies and strategies to further reduce the infection rates.

Many institutions have implemented bundles (e.g., products and processes) with the goal of improving compliance to all aspects of catheter care with variable success; however, a recurring theme in and outside of the U.S. is poor or non-existing cleaning of needleless connectors prior to each access. In addition, time and motion study shows documentation accounts for 35% of nurse practice time (e.g., about 2.5 hrs per shift), and that such documentation is often duplicative and error-prone, leading to further inefficiencies. Therefore, automating catheter care and maintenance documentation can free up nurses' time for patient care activities and reduce errors and inefficiencies associated therewith.

### SUMMARY

Accordingly, provided are improved systems, devices, products, apparatus, and/or methods for vascular access management, which may reduce time and/or errors associated with nurse documentation efforts and/or reduce a possibility of catheter-related failures, such as catheter related bloodstream infections (CRBSIs), central line associated bloodstream infections (CLABSIs), and/or the like.

According A to some non-limiting embodiments or aspects, provided is a method including: capturing, with one or more image capture devices, over a period of time, a plurality of images of an environment surrounding the one or more image capture devices; determining, with one or more processors, based on the plurality of images, a plurality of locations of a plurality of medical devices within the environment over the period of time and a plurality of types of the plurality of medical devices; and updating, with the one or more processors, based on the plurality of locations of the plurality of medical devices within the environment over the period of time and the plurality of types of the plurality of medical devices, a database including spatial relationships between the plurality of medical devices.

In some non-limiting embodiments or aspects, the method further includes: determining, with the one or more processors, based on the plurality of locations of the plurality of medical devices within the environment over the period of time and the plurality of types of the plurality of medical devices, at least one connection between two or more medical devices of the plurality of medical devices; and updating, with the one or more processors, based on the at least one connection determined between the two or more medical devices, the database.

In some non-limiting embodiments or aspects, the method further includes: determining, with the one or more processors, based on the plurality of images, a plurality of trajectories of the plurality of medical devices within the environment over the period of time; and updating, with the one or more processors, based on the plurality of trajectories of the plurality of medical devices within the environment over the period of time, the database.

In some non-limiting embodiments or aspects, the method further includes: determining, with the one or more processors, based on the plurality of images, a plurality of orientations of the plurality of medical devices within the environment over the period of time; and updating, with the one or more processors, based on the plurality of orientations of the plurality of medical devices within the environment over the period of time, the database.

In some non-limiting embodiments or aspects, the method further includes: determining, with the one or more processors, based on the plurality of locations of the plurality of medical devices within the environment over the period of time, the plurality of types of the plurality of medical devices, and the plurality of orientations of the plurality of medical devices within the environment over the period of time, at least one event including at least one connection between two or more medical devices of the plurality of medical devices that forms a fluid flow path through the two or more medical devices and a direction of a fluid flow in the fluid plow path through the two or more medical devices; and updating, with the one or more processors, based on the at least one connection determined between the two or more medical devices that forms the fluid flow path and the direction of the fluid flow path, the database.

In some non-limiting embodiments or aspects, the method further includes: determining, with the one or more processors, based on the plurality of locations of the plurality of medical devices within the environment over the period of time and the plurality of types of the plurality of medical devices, at least one event of the following events: (i) a reuse of a medical device including a disconnection of the medical device from at least one of a patient and another medical device in the environment and a reconnection of the medical device to the at least one of the patient and the another medical device in the environment, (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and updating, with the one or more processors, based on the determined event, the database including spatial relationships between the plurality of medical devices.

In some non-limiting embodiments or aspects, the type of the medical device includes a disposable medical device.

In some non-limiting embodiments or aspects, the at least one event includes the reuse of the medical device, and wherein the method further includes: in response to determining the reuse of the medical device, at least one of: providing, with the one or more processors, to a user device, an alert associated with the use of the medical device; and automatically controlling, with the one or more processors, at least one medical device to stop a flow of fluid in a fluid flow path including the medical device.

In some non-limiting embodiments or aspects, the plurality of types of the plurality of medical devices includes at least one medical device including at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

According to some non-limiting embodiments or aspects, provided is a system including: one or more image capture devices configured to capture, over a period of time, a plurality of images of an environment surrounding the one or more image capture devices; and one or more processors programmed and/or configured to: determine, based on the plurality of images, a plurality of locations of a plurality of medical devices within the environment over the period of time and a plurality of types of the plurality of medical devices; and update, based on the plurality of locations of the plurality of medical devices within the environment over the period of time and the plurality of types of the plurality of medical devices, a database including spatial relationships between the plurality of medical devices.

In some non-limiting embodiments or aspects, the one or more processors are further programmed and/or configured to: determine, based on the plurality of locations of the plurality of medical devices within the environment over the period of time and the plurality of types of the plurality of medical devices, at least one connection between two or more medical devices of the plurality of medical devices; and update, based on the at least one connection determined between the two or more medical devices, the database.

In some non-limiting embodiments or aspects, the one or more processors are further programmed and/or configured to: determine, based on the plurality of images, a plurality of trajectories of the plurality of medical devices within the environment over the period of time; and update, based on the plurality of trajectories of the plurality of medical devices within the environment over the period of time, the database.

In some non-limiting embodiments or aspects, the one or more processors are further programmed and/or configured to: determine, based on the plurality of images, a plurality of orientations of the plurality of medical devices within the environment over the period of time; and update, based on the plurality of orientations of the plurality of medical devices within the environment over the period of time, the database.

In some non-limiting embodiments or aspects, the one or more processors are further programmed and/or configured to: determine, based on the plurality of locations of the plurality of medical devices within the environment over the period of time, the plurality of types of the plurality of medical devices, and the plurality of orientations of the plurality of medical devices within the environment over the period of time, at least one event including at least one connection between two or more medical devices of the plurality of medical devices that forms a fluid flow path through the two or more medical devices and a direction of a fluid flow in the fluid plow path through the two or more medical devices; and update, based on the at least one connection determined between the two or more medical devices that forms the fluid flow path and the direction of the fluid flow path, the database.

In some non-limiting embodiments or aspects, the one or more processors are further programmed and/or configured to: determine, based on the plurality of locations of the plurality of medical devices within the environment over the period of time and the plurality of types of the plurality of medical devices, at least one event of the following events: (i) a reuse of a medical device including a disconnection of the medical device from at least one of a patient and another medical device in the environment and a reconnection of the medical device to the at least one of the patient and the another medical device in the environment, (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and update, based on the determined event, the database including spatial relationships between the plurality of medical devices.

In some non-limiting embodiments or aspects, the type of the medical device includes a disposable medical device.

In some non-limiting embodiments or aspects, the at least one event includes the reuse of the medical device, and wherein the one or more processors are further programmed and/or configured to: in response to determining the reuse of the medical device, at least one of: provide, to a user device, an alert associated with the use of the medical device; and automatically control at least one medical device to stop a flow of fluid in a fluid flow path including the medical device.

In some non-limiting embodiments or aspects, the plurality of types of the plurality of medical devices includes at least one medical device including at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

According to some non-limiting embodiments or aspects, provided is a method including: capturing, with one or more image capture devices, over a period of time, a plurality of images of an environment surrounding the one or more image capture devices; determining, with one or more processors, based on the plurality of images, a plurality of identifier elements within the environment over the period of time, wherein the plurality of identifier elements is associated with a plurality of medical devices, and wherein the plurality of identifier elements encapsulates a plurality of identifiers associated a plurality of types of the plurality of medical devices; determining, with the one or more processors, based on the plurality of identifier elements determined in the plurality of images, the plurality of types of the plurality of medical devices and a plurality of locations of the plurality of medical devices within the environment over the period of time; and updating, with the one or more processors, based on the plurality of types of the plurality of medical devices and the plurality of locations of the plurality of medical devices within the environment over the period of time, a database including spatial relationships between the plurality of medical devices.

In some non-limiting embodiments or aspects, the plurality of identifiers uniquely identify the plurality of medical devices from each other.

In some non-limiting embodiments or aspects, the plurality of types of the plurality of medical devices includes at least one identifier element that encapsulates at least one identifier associated with at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an medication delivery syringe, a caregiver glove, an IV fluid bag, a medication dispensing cabinet, an ultrasound device, a sharps collector, or any combination thereof.

In some non-limiting embodiments or aspects, the plurality of identifier elements includes at least one identifier element including at least one of the following types of identifier elements: a colored pattern, a reflective pattern, a fluorescent pattern, a predetermined shape and color, a LED pattern, a barcode, a fiducial marker, a hologram marker, or any combination thereof.

In some non-limiting embodiments or aspects, the plurality of identifier elements includes at least one identifier element including a fluorescent coating configured to emit light of a predetermined wavelength, and wherein the one or more image capture devices capture only the light of the predetermined wavelength in the plurality of images.

In some non-limiting embodiments or aspects, the plurality of identifier elements includes at least one identifier element including at least one LED configured to emit light of at least one predetermined wavelength in at least one pattern and/or at least one intensity, and wherein the one or more image capture devices capture only the light of the predetermined wavelength in the plurality of images.

In some non-limiting embodiments or aspects, the method further includes: determining, with the one or more processors, based on the at least one pattern and/or the at least one intensity of the emitted light of the at least one predetermined wavelength captured in the plurality of images, a type of a medical device associated with the at least one identifier element.

In some non-limiting embodiments or aspects, the plurality of identifier elements includes at least one identifier element including at least one color changing dye configured to change color over the period of time, and wherein the method further includes: determining, with the one or more processors, based on a change in the color of the color changing dye in the plurality of images, an amount of time associated with a use of a medical device associated with the at least one identifier element.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

In some non-limiting embodiments or aspects, the plurality of identifiers is further associated with a plurality of orientations of the plurality of medical devices, and wherein the method further includes: determining, with the one or more processors, based on the plurality of identifier elements determined in the plurality of images, the plurality of orientations of the plurality of medical devices within the environment over the period of time; and updating, based on the plurality of orientations of the plurality of medical devices within the environment over the period of time, the database.

According to some non-limiting embodiments or aspects, provided is a system including: a plurality of identifier elements associated with a plurality of medical devices, wherein the plurality of identifier elements encapsulates a plurality of identifiers associated a plurality of types of the plurality of medical devices; one or more image capture devices configured to capture, over a period of time, a plurality of images of an environment surrounding the one or more image capture devices; and one or more processors programmed and/or configured to: determine, based on the plurality of images, the plurality of identifier elements within the environment over the period of time; determine, based on the plurality of identifier elements determined in the plurality of images, the plurality of types of the plurality of medical devices and a plurality of locations of the plurality of medical devices within the environment over the period of time; and update, based on the plurality of types of the plurality of medical devices and the plurality of locations of the plurality of medical devices within the environment over the period of time, a database including spatial relationships between the plurality of medical devices.

In some non-limiting embodiments or aspects, the plurality of identifiers uniquely identify the plurality of medical devices from each other.

In some non-limiting embodiments or aspects, the plurality of types of the plurality of medical devices includes at least one identifier element that encapsulates at least one identifier associated with at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an medication delivery syringe, a caregiver glove, an IV fluid bag, a medication dispensing cabinet, an ultrasound device, a sharps collector, or any combination thereof.

In some non-limiting embodiments or aspects, the plurality of identifier elements includes at least one identifier element including at least one of the following types of identifier elements: a colored pattern, a reflective pattern, a fluorescent pattern, a predetermined shape and color, a LED pattern, a barcode, a fiducial marker, a hologram marker, or any combination thereof.

In some non-limiting embodiments or aspects, the plurality of identifier elements includes at least one identifier element including a fluorescent coating configured to emit light of a predetermined wavelength, wherein the one or more image capture devices are configured to capture only the light of the predetermined wavelength in the plurality of images.

In some non-limiting embodiments or aspects, the plurality of identifier elements includes at least one identifier element including at least one LED configured to emit light of at least one predetermined wavelength in at least one pattern and/or at least one intensity, wherein the one or more image capture devices are configured to capture only the light of the predetermined wavelength in the plurality of images.

In some non-limiting embodiments or aspects, the one or more processors are further programmed and/or configured to determine, based on the at least one pattern and/or the at least one intensity of the emitted light of the at least one predetermined wavelength captured in the plurality of images, a type of a medical device associated with the at least one identifier element.

In some non-limiting embodiments or aspects, the plurality of identifier elements includes at least one identifier element including at least one color changing dye configured to change color over the period of time, wherein the one or more processors are further programmed and/or configured to determine, based on a change in the color of the color changing dye in the plurality of images, an amount of time associated with a use of a medical device associated with the at least one identifier element.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

In some non-limiting embodiments or aspects, the plurality of identifiers is further associated with a plurality of orientations of the plurality of medical devices, and wherein the one or more processors are further programmed and/or configured to: determine, based on the plurality of identifier elements determined in the plurality of images, the plurality of orientations of the plurality of medical devices within the environment over the period of time; and update, based on the plurality of orientations of the plurality of medical devices within the environment over the period of time, the database.

According to some non-limiting embodiments or aspects, provided is a method including: capturing, with one or more image capture devices, over a period of time, a plurality of images of an environment surrounding the one or more image capture devices; determining, with one or more processors, based on the plurality of images, a plurality of locations of a plurality of medical devices within the environment over the period of time and a plurality of types of the plurality of medical devices; determining, with the one or more processors, based on the plurality of locations of the plurality of medical devices within the environment over the period of time, a plurality of distances between the plurality of medical devices over the period of time; determining, with the one or more processors, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and updating, with the one or more processors, based on the at least one determined event, a database including current connections between the plurality of medical devices.

In some non-limiting embodiments or aspects, the first medical device includes at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

In some non-limiting embodiments or aspects, the method further includes: determining, with the one or more processors, based on the plurality of distances between the plurality of medical devices over the period of time and the plurality of types of the plurality of medical devices, one of the following further events: (i) a reuse of the first medical device including a disconnection of the first medical device from the second medical device in the environment and a reconnection of the first medical device to the second medical device in the environment, (ii) a replacement of the first medical device with a new medical device of a same type as the first medical device including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and updating, with the one or more processors, based on the further determined event, the database including the spatial relationships between the plurality of medical devices.

In some non-limiting embodiments or aspects, the method further includes: determining, with the one or more processors, based on the plurality of images, a plurality of orientations of the plurality of medical devices within the environment over the period of time, wherein determining the at least one event is further based on the plurality of orientations of the plurality of medical devices within the environment over the period of time.

In some non-limiting embodiments or aspects, the database further includes directions of fluid flows in fluid flow paths through the current connections, wherein the at least one event includes the connection of the first medical device of the plurality of medical devices to the second medical device of the plurality of medical devices, and wherein the method further includes: determining, with the one or more processors, based on an orientation of the first medical device and an orientation of the second medical device, a direction of a fluid flow in a fluid plow path through the first medical device and the second medical device; and updating, with the one or more processors, based on the direction of the fluid flow in the fluid plow path through the first medical device and the second medical device, the database.

In some non-limiting embodiments or aspects, the first medical device includes at least one of a disinfectant cap and a disinfectant swab such that the connection of the first medical device of the plurality of medical devices to the second medical device of the plurality of medical devices does not form a fluid flow path through the first medical device and the second medical device.

In some non-limiting embodiments or aspects, determining the at least one event further determines a probability associated with the at least one event.

In some non-limiting embodiments or aspects, the method further includes: delaying, with the one or more processors, determining the plurality of distances between the plurality of medical devices over the period of time and determining the at least one event until a location of at least one of the first medical device and the second medical device changes in the plurality of images over the period of time.

In some non-limiting embodiments or aspects, the method further includes: in response to determining the at least one event, at least one of: providing, with the one or more processors, to a user device, an alert associated with the at least one event; and automatically controlling, with the one or more processors, at least one medical device to stop a flow of fluid in a fluid flow path including the medical device.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

According to some non-limiting embodiments or aspects, provided is a system including: one or more image capture devices configured to capture, over a period of time, a plurality of images of an environment surrounding the one or more image capture devices; one or more processors programmed and/or configured to: determine, based on the plurality of images, a plurality of locations of a plurality of medical devices within the environment over the period of time and a plurality of types of the plurality of medical devices; determine, based on the plurality of locations of the plurality of medical devices within the environment over the period of time, a plurality of distances between the plurality of medical devices over the period of time; determine, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and update, based on the at least one determined event, a database including current connections between the plurality of medical devices.

In some non-limiting embodiments or aspects, the first medical device includes at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

In some non-limiting embodiments or aspects, the one or more processors are further programmed and/or configured to: determine, based on the plurality of distances between the plurality of medical devices over the period of time and the plurality of types of the plurality of medical devices, one of the following further events: (i) a reuse of the first medical device including a disconnection of the first medical device from the second medical device in the environment and a reconnection of the first medical device to the second medical device in the environment, (ii) a replacement of the first medical device with a new medical device of a same type as the first medical device including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and update, based on the further determined event, the database including the spatial relationships between the plurality of medical devices.

In some non-limiting embodiments or aspects, the one or more processors are further programmed and/or configured to: determine, based on the plurality of images, a plurality of orientations of the plurality of medical devices within the environment over the period of time, wherein determining the at least one event is further based on the plurality of orientations of the plurality of medical devices within the environment over the period of time.

In some non-limiting embodiments or aspects, the database further includes directions of fluid flows in fluid flow paths through the current connections, wherein the at least one event includes the connection of the first medical device of the plurality of medical devices to the second medical device of the plurality of medical devices, and wherein the one or more processors are further programmed and/or configured to: determine, based on an orientation of the first medical device and an orientation of the second medical device, a direction of a fluid flow in a fluid plow path through the first medical device and the second medical device; and update, based on the direction of the fluid flow in the fluid plow path through the first medical device and the second medical device, the database.

In some non-limiting embodiments or aspects, the first medical device includes at least one of a disinfectant cap and a disinfectant swab such that the connection of the first medical device of the plurality of medical devices to the second medical device of the plurality of medical devices does not form a fluid flow path through the first medical device and the second medical device.

In some non-limiting embodiments or aspects, determining the at least one event further determines a probability associated with the at least one event.

In some non-limiting embodiments or aspects, the one or more processors are further programmed and/or configured to: delay determining the plurality of distances between the plurality of medical devices over the period of time and determining the at least one event until a location of at least one of the first medical device and the second medical device changes in the plurality of images over the period of time.

In some non-limiting embodiments or aspects, the one or more processors are further programmed and/or configured to: in response to determining the at least one event, at least one of: provide, to a user device, an alert associated with the at least one event; and automatically control at least one medical device to stop a flow of fluid in a fluid flow path including the medical device.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

According to some non-limiting embodiments or aspects, provided is a method including: capturing, with one or more image capture devices, over a period of time, a plurality of images of an environment surrounding the one or more image capture devices; determining, with one or more processors, based on the plurality of images, a first identifier element associated with a medical device and a second identifier element associated with a glove of a caregiver, wherein the first identifier element encapsulates a first identifier associated with the medical device, and wherein the second identifier element encapsulates a second identifier associated with the glove of the caregiver; determining, with the one or more processors, based on the first identifier element in the plurality of images, the medical device and a location of the medical device within the environment over the period of time; determining, with the one or more processors, based on the second identifier element in the plurality of images, the glove of the caregiver and a location of the glove of the caregiver within the environment over the period of time; determining, with the one or more processors, based on the location of the medical device within the environment over the period of time and the location of the glove of the caregiver within the environment over the period of time and the location, at least one event associated with the medical device; and updating, with the one or more processors, based on the at least one determined event, a database including events associated with the environment.

In some non-limiting embodiments or aspects, the method further includes: determining, with the one or more processors, based on the plurality of images, a location of a further medical device within the environment over the period of time and a type of the further medical device; and determining, with the one or more processors, based on the location of the further medical device over the period of time and at least one of (i) the location of medical device within the environment over the period of time and (ii) the location of the glove of the caregiver within the environment over the period of time, a linear distance change and an angular distance change between the further medical device and the at least one of the medical device and the glove of the caregiver over the period of time, wherein determining the at least one event is further based on the linear distance change and the angular distance change between the medical device and the glove of the caregiver.

In some non-limiting embodiments or aspects, the at least one event includes a scrubbing or disinfecting event.

In some non-limiting embodiments or aspects, the method further includes: determining, with the one or more processors, based on the on the linear distance change and the angular distance change between the medical device and the glove of the caregiver over the period of time, a duration of the scrubbing action.

In some non-limiting embodiments or aspects, the further medical device includes a disinfectant swab or wipe.

In some non-limiting embodiments or aspects, the medical device includes a needless connector.

In some non-limiting embodiments or aspects, the at least one event includes a catheter dressing change event, and wherein the medical device includes a catheter dressing.

In some non-limiting embodiments or aspects, the second identifier element includes a predetermined color of the glove of the caregiver.

In some non-limiting embodiments or aspects, the first identifier element includes at least one of: a colored pattern, a reflective pattern, a fluorescent pattern, a LED pattern, a barcode, a fiducial marker, a hologram marker, or any combination thereof.

In some non-limiting embodiments or aspects, the method further includes: in response to determining the at least one event, at least one of: providing, with the one or more processors, to a user device, an alert associated with the at least one event; and automatically controlling, with the one or more processors, at least one medical device to stop a flow of fluid in a fluid flow path including the medical device.

According to some non-limiting embodiments or aspects, provided is a system including: a first identifier element associated with a medical device, wherein the first identifier element encapsulates a first identifier associated with the medical device; a second identifier element associated with a glove of a caregiver, wherein the second identifier element encapsulates a second identifier associated with the glove of the caregiver; one or more image capture devices configured to capture, over a period of time, a plurality of images of an environment surrounding the one or more image capture devices; and one or more processors programmed and/or configured to: determine, based on the plurality of images, the first identifier element associated with the medical device and the second identifier element associated with the glove of a caregiver; determine, based on the first identifier element in the plurality of images, the medical device and a location of the medical device within the environment over the period of time; determine, based on the second identifier element in the plurality of images, the glove of the caregiver and a location of the glove of the caregiver within the environment over the period of time; determine, based on the location of the medical device within the environment over the period of time and the location of the glove of the caregiver within the environment over the period of time and the location, at least one event associated with the medical device; and update, based on the at least one determined event, a database including events associated with the environment.

In some non-limiting embodiments or aspects, the one or more processors are further programmed and/or configured to: determine, based on the plurality of images, a location of a further medical device within the environment over the period of time and a type of the further medical device; and determine, based on the location of the further medical device over the period of time and at least one of (i) the location of medical device within the environment over the period of time and (ii) the location of the glove of the caregiver within the environment over the period of time, a linear distance change and an angular distance change between the further medical device and the at least one of the medical device and the glove of the caregiver over the period of time, wherein determining the at least one event is further based on the linear distance change and the angular distance change between the medical device and the glove of the caregiver.

In some non-limiting embodiments or aspects, the at least one event includes a scrubbing or disinfecting event.

In some non-limiting embodiments or aspects, the one or more processors are further programmed and/or configured to: determine, based on the on the linear distance change and the angular distance change between the medical device and the glove of the caregiver over the period of time, a duration of the scrubbing action.

In some non-limiting embodiments or aspects, the further medical device includes a disinfectant swab or wipe.

In some non-limiting embodiments or aspects, the medical device includes a needless connector.

In some non-limiting embodiments or aspects, the at least one event includes a catheter dressing change event, and wherein the medical device includes a catheter dressing.

In some non-limiting embodiments or aspects, the second identifier element includes a predetermined color of the glove of the caregiver.

In some non-limiting embodiments or aspects, the first identifier element includes at least one of: a colored pattern, a reflective pattern, a fluorescent pattern, a LED pattern, a barcode, a fiducial marker, a hologram marker, or any combination thereof.

In some non-limiting embodiments or aspects, the one or more processors are further programmed and/or configured to: in response to determining the at least one event, at least one of: provide, to a user device, an alert associated with the at least one event; and automatically control, at least one medical device to stop a flow of fluid in a fluid flow path including the medical device.

According to some non-limiting embodiments or aspects, provided is a method including: capturing, with one or more image capture devices, over a period of time, a plurality of images of an environment surrounding the one or more image capture devices; determining, with one or more processors, based on the plurality of images, a location of a plunger of a syringe relative to a barrel of the syringe in the environment over the period of time; determining, with the one or more processors, based on the location of the plunger of the syringe relative to the barrel of the syringe over the period of time, at least one fluid delivery from the syringe; and updating, with the one or more processors, based on the at least one determined fluid delivery, a database including fluid deliveries associated with the environment.

In some non-limiting embodiments or aspects, determining the least one fluid delivery further includes determining, based on the location of the plunger of the syringe relative to the barrel of the syringe over the period of time, at least one of an amount of fluid delivered by the at least one fluid delivery and a fluid flow rate of the at least one fluid delivery.

In some non-limiting embodiments or aspects, the method further includes: determining, with the one or more processors, based on the fluid flow rate of the at least one fluid delivery, a type of the fluid associated with the at least one fluid delivery.

In some non-limiting embodiments or aspects, the method further includes: determining, with the one or more processors, based on the plurality of images, a location of a glove of a caregiver within the environment over the period of time; and determining, with the one or more processors, based on the location of the glove of the caregiver within the environment over the period of time, a flush technique associated with the at least one fluid delivery, wherein the flush technique includes a pulsatile flush or a continuous flush.

In some non-limiting embodiments or aspects, determining the location of the plunger of the syringe relative to the barrel of the syringe over the period of time further includes determining, based on the plurality of images, a first identifier element associated with the plunger of the syringe and a second identifier element associated with the barrel of the syringe, wherein the location of the plunger of a syringe relative to a barrel of the syringe in the environment over the period of time is determined based on the first identifier element in the plurality of images and the second identifier element in the plurality of images.

In some non-limiting embodiments or aspects, determining the location of the plunger of the syringe relative to the barrel of the syringe in the environment over the period of time includes determining, based on the first identifier element and the second identifier element in the plurality of images, a distance between the first identifier element and the second identifier element as a distance between the plunger and the barrel of the syringe.

In some non-limiting embodiments or aspects, the first identifier element and the second identifier element include at least one of the following: a colored pattern, a reflective pattern, a fluorescent pattern, a predetermined shape and color, a LED pattern, a barcode, a fiducial marker, a hologram marker, or any combination thereof.

In some non-limiting embodiments or aspects, the method further includes: determining, with the one or more processors, based on the plurality of images, a color of a fluid contained in the syringe; and determining, with the one or more processors, based on the color of the fluid, a type of the fluid associated with the at least one fluid delivery.

In some non-limiting embodiments or aspects, the plurality of images include at least one infrared (IR) image, and wherein the method further includes: determining, with the one or more processors, based on the at least one IR image, a temperature of a fluid contained in the syringe; and determining, with the one or more processors, based on the temperature of the fluid contained in the syringe, a type of the fluid associated with the at least one fluid delivery.

In some non-limiting embodiments or aspects, the plurality of images include at least one infrared (IR) image, and wherein the method further includes: determining, with the one or more processors, based on the at least one IR image, a temperature of a fluid contained in the syringe; comparing, with the one or more processors, the temperature of the fluid contained in the syringe to a threshold temperature associated with the fluid; and in response to determining that the temperature of the fluid contained in the syringe satisfies the threshold temperature, automatically controlling, with the one or more processors, at least one medical device to stop the at least one fluid delivery from the syringe.

According to some non-limiting embodiments or aspects, provided is a system including: one or more image capture devices configured to capture, over a period of time, a plurality of images of an environment surrounding the one or more image capture devices; and one or more processors programmed and/or configured to: determine, based on the plurality of images, a location of a plunger of a syringe relative to a barrel of the syringe in the environment over the period of time; determine, based on the location of the plunger of the syringe relative to the barrel of the syringe over the period of time, at least one fluid delivery from the syringe; and update, based on the at least one determined fluid delivery, a database including fluid deliveries associated with the environment.

In some non-limiting embodiments or aspects, the one or more processors are further programmed and/or configured to: determine, based on the location of the plunger of the syringe relative to the barrel of the syringe over the period of time, at least one of an amount of fluid delivered by the at least one fluid delivery and a fluid flow rate of the at least one fluid delivery.

In some non-limiting embodiments or aspects, the one or more processors are further programmed and/or configured to: determine, based on the fluid flow rate of the at least one fluid delivery, a type of the fluid associated with the at least one fluid delivery.

In some non-limiting embodiments or aspects, the one or more processors are further programmed and/or configured to: determine, based on the plurality of images, a location of a glove of a caregiver within the environment over the period of time; and determine, based on the location of the glove of the caregiver within the environment over the period of time, a flush technique associated with the at least one fluid delivery, wherein the flush technique includes a pulsatile flush or a continuous flush.

In some non-limiting embodiments or aspects, the one or more processors are programmed and/or configured to determine the location of the plunger of the syringe relative to the barrel of the syringe over the period of time by determining, based on the plurality of images, a first identifier element associated with the plunger of the syringe and a second identifier element associated with the barrel of the syringe, wherein the location of the plunger of a syringe relative to a barrel of the syringe in the environment over the period of time is determined based on the first identifier element in the plurality of images and the second identifier element in the plurality of images.

In some non-limiting embodiments or aspects, the one or more processors are programmed and/or configured to determine the location of the plunger of the syringe relative to the barrel of the syringe in the environment over the period of time by determining, based on the first identifier element and the second identifier element in the plurality of images, a distance between the first identifier element and the second identifier element as a distance between the plunger and the barrel of the syringe.

In some non-limiting embodiments or aspects, the first identifier element and the second identifier element include at least one of the following: a colored pattern, a reflective pattern, a fluorescent pattern, a predetermined shape and color, a LED pattern, a barcode, a fiducial marker, a hologram marker, or any combination thereof.

In some non-limiting embodiments or aspects, the one or more processors are further programmed and/or configured to: determine, based on the plurality of images, a color of a fluid contained in the syringe; and determine, based on the color of the fluid, a type of the fluid associated with the at least one fluid delivery.

In some non-limiting embodiments or aspects, the plurality of images include at least one infrared (IR) image, and wherein the one or more processors are further programmed and/or configured to: determine, based on the at least one IR image, a temperature of a fluid contained in the syringe; and determine, based on the temperature of the fluid contained in the syringe, a type of the fluid associated with the at least one fluid delivery.

In some non-limiting embodiments or aspects, the plurality of images include at least one infrared (IR) image, and wherein the one or more processors are further programmed and/or configured to: determine, based on the at least one IR image, a temperature of a fluid contained in the syringe; compare, the temperature of the fluid contained in the syringe to a threshold temperature associated with the fluid; and in response to determining that the temperature of the fluid contained in the syringe satisfies the threshold temperature, automatically control, at least one medical device to stop the at least one fluid delivery from the syringe.

According to some non-limiting embodiments or aspects, provided is a method including: capturing, with one or more image capture devices, over a period of time, a plurality of images of an environment surrounding the one or more image capture devices; determining, with one or more processors, based on the plurality of images, a state of a package containing a medical device over the period of time; determining, with the one or more processors, based on the state of the package over the period of time, whether the medical device is removed from the package; and updating, with the one or more processors, based on a determination that the medical device is removed from the package, a database including medical devices in the environment.

In some non-limiting embodiments or aspects, the package includes a removable first layer covering a second layer, wherein the first layer including a first color, wherein the second layer includes a second color different than the first color, and wherein removal of the first layer from the package reveals the second layer.

In some non-limiting embodiments or aspects, the first layer is at least partially transparent.

In some non-limiting embodiments or aspects, a color of the package is configured to change when exposed to air.

In some non-limiting embodiments or aspects, a portion of the package is transparent such that the medical device contained within the package is visible through the transparent portion of the package.

In some non-limiting embodiments or aspects, the package is associated with a first identifier element, wherein the medical device is associated with a second identifier element different than the first identifier element, and wherein the state of the package is determined based on a location of the first identifier element with respect to a location of the second identifier element.

In some non-limiting embodiments or aspects, the method further includes: determining, based on the plurality of images, a location of a glove of a caregiver within the environment over the period of time with respect to a location of the package, wherein the state of the package is determined based on the location of the glove of the caregiver within the environment over the period of time with respect to the location of the package.

In some non-limiting embodiments or aspects, the package includes a removable first layer covering a second layer, wherein the removable first layer includes a first identifier element, wherein the second layer includes a second identifier element, wherein the removable first layer is at least partially transparent, and wherein determining the state of the package further includes: determining, based on the plurality of images, a distance between the first identifier element and the second identifier element; and determining, based on the distance between the first identifier element and the second identifier element, whether the package is faulty; and in response to determining that the package is faulty, providing, to a user device, an alert associated with the faulty package.

In some non-limiting embodiments or aspects, the method further includes: determining, with the one or more processors, based on the plurality of images, a plurality of locations of a plurality of medical devices within the environment over the period of time and a plurality of types of the plurality of medical devices; and determining, with the one or more processors, based on the plurality of locations of the plurality of medical devices within the environment over the period of time, the plurality of types of the plurality of medical devices, and the state of the package over the period of time, at least one event of the following events: (i) a reuse of the medical device including a connection of the medical device to two or more medical devices in the environment over the period of time, (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device.

In some non-limiting embodiments or aspects, the medical device includes a disinfectant swab or wipe.

According to some non-limiting embodiments or aspects, provided is a system including: a package containing a medical device; one or more image capture devices configured to capture, over a period of time, a plurality of images of an environment surrounding the one or more image capture devices; and one or more processors programmed and/or configured to: determine, based on the plurality of images, a state of the package over the period of time; determine, based on the state of the package over the period of time, whether the medical device is removed from the package; and update, based on a determination that the medical device is removed from the package, a database including medical devices in the environment.

In some non-limiting embodiments or aspects, the package includes a removable first layer covering a second layer, wherein the first layer including a first color, wherein the second layer includes a second color different than the first color, and wherein removal of the first layer from the package reveals the second layer.

In some non-limiting embodiments or aspects, the first layer is at least partially transparent.

In some non-limiting embodiments or aspects, a color of the package is configured to change when exposed to air.

In some non-limiting embodiments or aspects, a portion of the package is transparent such that the medical device contained within the package is visible through the transparent portion of the package.

In some non-limiting embodiments or aspects, the package is associated with a first identifier element, wherein the medical device is associated with a second identifier element different than the first identifier element, and wherein the state of the package is determined based on a location of the first identifier element with respect to a location of the second identifier element.

In some non-limiting embodiments or aspects, the one or more processors are further programmed and/or configured to determine, based on the plurality of images, a location of a glove of a caregiver within the environment over the period of time with respect to a location of the package, wherein the state of the package is determined based on the location of the glove of the caregiver within the environment over the period of time with respect to the location of the package.

In some non-limiting embodiments or aspects, the package includes a removable first layer covering a second layer, wherein the removable first layer includes a first identifier element, wherein the second layer includes a second identifier element, wherein the removable first layer is at least partially transparent, and wherein the one or more processors are further programmed and/or configured to: determine, based on the plurality of images, a distance between the first identifier element and the second identifier element; and determine, based on the distance between the first identifier element and the second identifier element, whether the package is faulty; and in response to determining that the package is faulty, provide, to a user device, an alert associated with the faulty package.

In some non-limiting embodiments or aspects, the one or more processors are further programmed and/or configured to: determine, based on the plurality of images, a plurality of locations of a plurality of medical devices within the environment over the period of time and a plurality of types of the plurality of medical devices; and determine, based on the plurality of locations of the plurality of medical devices within the environment over the period of time, the plurality of types of the plurality of medical devices, and the state of the package over the period of time, at least one event of the following events: (i) a reuse of the medical device including a connection of the medical device to two or more medical devices in the environment over the period of time, (ii) a replacement of the medical device with a new medical device of a same type as the medical device in the environment, and (iii) a replacement of the medical device with different medical device of a different type than the medical device in the environment.

In some non-limiting embodiments or aspects, the medical device includes a disinfectant swab or wipe.

According to some non-limiting embodiments or aspects, provide is a method including: capturing, with one or more image capture devices, over a period of time, a plurality of images of an environment surrounding the one or more image capture devices; obtaining, with one or more processors, from a data source other than the one or more image capture devices, auxiliary data associated with at least one of: at least one image of the environment during the period of time and audio recorded in the environment during the period of time; determining, with the one or more processors, based on the plurality of images and the auxiliary data, a location of a medical device within the environment over the period of time; and updating, with the one or more processors, based on the location of the medical device within the environment over the period of time, a database including a spatial relationship of the medical device and at least one other medical device.

In some non-limiting embodiments or aspects, the medical device is not visible in at least a portion of the plurality of images.

In some non-limiting embodiments or aspects, the medical device includes a catheter, and wherein the auxiliary data includes at least one of: a tubing type of the catheter, a size of the catheter, a shape of the catheter, and a location of a catheter insertion site of the catheter on a patient.

In some non-limiting embodiments or aspects, the auxiliary data is associated with the audio recorded in the environment during the period of time.

In some non-limiting embodiments or aspects, the audio includes a predetermined signal associated with the medical device.

In some non-limiting embodiments or aspects, the medical device includes an infusion pump.

In some non-limiting embodiments or aspects, the method further includes: determining, with the one or more processors, based on the plurality of images and the auxiliary data, a plurality of locations of a plurality of medical devices within the environment over the period of time and a plurality of types of the plurality of medical devices, wherein the plurality of medical devices includes the medical device and the at least one other medical device, wherein the medical device and the at least one other medical device are configured to emit a predetermined audible signal when connected; and determining, with the one or more processors, based on the plurality of locations of the plurality of medical devices within the environment over the period of time and the plurality of types of the plurality of medical devices, at least one connection between two or more medical devices of the plurality of medical devices; and updating, with the one or more processors, based on the at least one connection determined between the two or more medical devices, the database.

In some non-limiting embodiments or aspects, the audio includes the predetermined audible signal associated with connection of the medical device and the at least one other medical device.

In some non-limiting embodiments or aspects, the medical device includes at least one of: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

According to some non-limiting embodiments or aspects, provided is system including: one or more image capture devices configured to capture, over a period of time, a plurality of images of an environment surrounding the one or more image capture devices; and one or more processors programmed and/or configured to: obtain, from a data source other than the one or more image capture devices, auxiliary data associated with at least one of: at least one image of the environment during the period of time and audio recorded in the environment during the period of time; determine, based on the plurality of images and the auxiliary data, a location of a medical device within the environment over the period of time; and update, based on the location of the medical device within the environment over the period of time, a database including a spatial relationship of the medical device and at least one other medical device.

In some non-limiting embodiments or aspects, the medical device is not visible in at least a portion of the plurality of images.

In some non-limiting embodiments or aspects, the medical device includes a catheter, and wherein the auxiliary data includes at least one of: a tubing type of the catheter, a size of the catheter, a shape of the catheter, and a location of a catheter insertion site of the catheter on a patient.

In some non-limiting embodiments or aspects, the auxiliary data is associated with the audio recorded in the environment during the period of time.

In some non-limiting embodiments or aspects, the audio includes a predetermined signal associated with the medical device.

In some non-limiting embodiments or aspects, the medical device includes an infusion pump.

In some non-limiting embodiments or aspects, the one or more processors are further programmed and/or configured to: determine, based on the plurality of images and the auxiliary data, a plurality of locations of a plurality of medical devices within the environment over the period of time and a plurality of types of the plurality of medical devices, wherein the plurality of medical devices includes the medical device and the at least one other medical device, wherein the medical device and the at least one other medical device are configured to emit a predetermined audible signal when connected; and determine, based on the plurality of locations of the plurality of medical devices within the environment over the period of time and the plurality of types of the plurality of medical devices, at least one connection between two or more medical devices of the plurality of medical devices; and update, based on the at least one connection determined between the two or more medical devices, the database.

In some non-limiting embodiments or aspects, the audio includes the predetermined audible signal associated with connection of the medical device and the at least one other medical device.

In some non-limiting embodiments or aspects, the medical device includes at least one of: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

In some non-limiting embodiments or aspects, the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

Further non-limiting embodiments or aspects are set forth in the following numbered clauses:

Clause 1a. A method comprising: capturing, with one or more image capture devices, over a period of time, a plurality of images of an environment surrounding the one or more image capture devices; determining, with one or more processors, based on the plurality of images, a plurality of locations of a plurality of medical devices within the environment over the period of time and a plurality of types of the plurality of medical devices; and updating, with the one or more processors, based on the plurality of locations of the plurality of medical devices within the environment over the period of time and the plurality of types of the plurality of medical devices, a database including spatial relationships between the plurality of medical devices.

Clause 2a. The method of clause 1a, further comprising: determining, with the one or more processors, based on the plurality of locations of the plurality of medical devices within the environment over the period of time and the plurality of types of the plurality of medical devices, at least one connection between two or more medical devices of the plurality of medical devices; and updating, with the one or more processors, based on the at least one connection determined between the two or more medical devices, the database.

Clause 3a. The method of any of clauses 1a and 2a, further comprising: determining, with the one or more processors, based on the plurality of images, a plurality of trajectories of the plurality of medical devices within the environment over the period of time; and updating, with the one or more processors, based on the plurality of trajectories of the plurality of medical devices within the environment over the period of time, the database.

Clause 4a. The method of any of clauses 1a-3a further comprising: determining, with the one or more processors, based on the plurality of images, a plurality of orientations of the plurality of medical devices within the environment over the period of time; and updating, with the one or more processors, based on the plurality of orientations of the plurality of medical devices within the environment over the period of time, the database.

Clause 5a. The method of any of clauses 1a-4a further comprising: determining, with the one or more processors, based on the plurality of locations of the plurality of medical devices within the environment over the period of time, the plurality of types of the plurality of medical devices, and the plurality of orientations of the plurality of medical devices within the environment over the period of time, at least one event including at least one connection between two or more medical devices of the plurality of medical devices that forms a fluid flow path through the two or more medical devices and a direction of a fluid flow in the fluid plow path through the two or more medical devices; and updating, with the one or more processors, based on the at least one connection determined between the two or more medical devices that forms the fluid flow path and the direction of the fluid flow path, the database.

Clause 6a. The method of any of clauses 1a-5a, further comprising: determining, with the one or more processors, based on the plurality of locations of the plurality of medical devices within the environment over the period of time and the plurality of types of the plurality of medical devices, at least one event of the following events: (i) a reuse of a medical device including a disconnection of the medical device from at least one of a patient and another medical device in the environment and a reconnection of the medical device to the at least one of the patient and the another medical device in the environment, (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and updating, with the one or more processors, based on the determined event, the database including spatial relationships between the plurality of medical devices.

Clause 7a. The method of any of clauses 1a-6a, wherein the type of the medical device includes a disposable medical device.

Clause 8a. The method of any of clauses 1a-7a, wherein the at least one event includes the reuse of the medical device, and wherein the method further comprises: in response to determining the reuse of the medical device, at least one of: providing, with the one or more processors, to a user device, an alert associated with the use of the medical device; and automatically controlling, with the one or more processors, at least one medical device to stop a flow of fluid in a fluid flow path including the medical device.

Clause 9a. The method of any of clauses 1a-8a, wherein the plurality of types of the plurality of medical devices includes at least one medical device including at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 10a. The method of any of clauses 1a-9a, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

Clause 11a. A system comprising: one or more image capture devices configured to capture, over a period of time, a plurality of images of an environment surrounding the one or more image capture devices; and one or more processors programmed and/or configured to: determine, based on the plurality of images, a plurality of locations of a plurality of medical devices within the environment over the period of time and a plurality of types of the plurality of medical devices; and update, based on the plurality of locations of the plurality of medical devices within the environment over the period of time and the plurality of types of the plurality of medical devices, a database including spatial relationships between the plurality of medical devices.

Clause 12a. The system of clause 11a, wherein the one or more processors are further programmed and/or configured to: determine, based on the plurality of locations of the plurality of medical devices within the environment over the period of time and the plurality of types of the plurality of medical devices, at least one connection between two or more medical devices of the plurality of medical devices; and update, based on the at least one connection determined between the two or more medical devices, the database.

Clause 13a. The system of any of clauses 11a and 12a, wherein the one or more processors are further programmed and/or configured to: determine, based on the plurality of images, a plurality of trajectories of the plurality of medical devices within the environment over the period of time; and update, based on the plurality of trajectories of the plurality of medical devices within the environment over the period of time, the database.

Clause 14a. The system of any of clauses 11a-13a, wherein the one or more processors are further programmed and/or configured to: determine, based on the plurality of images, a plurality of orientations of the plurality of medical devices within the environment over the period of time; and update, based on the plurality of orientations of the plurality of medical devices within the environment over the period of time, the database.

Clause 15a. The system of any of clauses 11a-14a, wherein the one or more processors are further programmed and/or configured to: determine, based on the plurality of locations of the plurality of medical devices within the environment over the period of time, the plurality of types of the plurality of medical devices, and the plurality of orientations of the plurality of medical devices within the environment over the period of time, at least one event including at least one connection between two or more medical devices of the plurality of medical devices that forms a fluid flow path through the two or more medical devices and a direction of a fluid flow in the fluid plow path through the two or more medical devices; and update, based on the at least one connection determined between the two or more medical devices that forms the fluid flow path and the direction of the fluid flow path, the database.

Clause 16a. The system of any of clauses 11a-15a, wherein the one or more processors are further programmed and/or configured to: determine, based on the plurality of locations of the plurality of medical devices within the environment over the period of time and the plurality of types of the plurality of medical devices, at least one event of the following events: (i) a reuse of a medical device including a disconnection of the medical device from at least one of a patient and another medical device in the environment and a reconnection of the medical device to the at least one of the patient and the another medical device in the environment, (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and update, based on the determined event, the database including spatial relationships between the plurality of medical devices.

Clause 17a. The system of any of clauses 11a-16a, wherein the type of the medical device includes a disposable medical device.

Clause 18a. The system of any of clauses 11a-17a, wherein the at least one event includes the reuse of the medical device, and wherein the one or more processors are further programmed and/or configured to: in response to determining the reuse of the medical device, at least one of: provide, to a user device, an alert associated with the use of the medical device; and automatically control at least one medical device to stop a flow of fluid in a fluid flow path including the medical device.

Clause 19a. The system of any of clauses 11a-18a, wherein the plurality of types of the plurality of medical devices includes at least one medical device including at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 20a. The system of any of clauses 11a-19a, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

Clause 1b. A method comprising: capturing, with one or more image capture devices, over a period of time, a plurality of images of an environment surrounding the one or more image capture devices; determining, with one or more processors, based on the plurality of images, a plurality of identifier elements within the environment over the period of time, wherein the plurality of identifier elements is associated with a plurality of medical devices, and wherein the plurality of identifier elements encapsulates a plurality of identifiers associated a plurality of types of the plurality of medical devices; determining, with the one or more processors, based on the plurality of identifier elements determined in the plurality of images, the plurality of types of the plurality of medical devices and a plurality of locations of the plurality of medical devices within the environment over the period of time; and updating, with the one or more processors, based on the plurality of types of the plurality of medical devices and the plurality of locations of the plurality of medical devices within the environment over the period of time, a database including spatial relationships between the plurality of medical devices.

Clause 2b. The method of clause 1b, wherein the plurality of identifiers uniquely identify the plurality of medical devices from each other.

Clause 3b. The method of any of clauses 1b and 2b, wherein the plurality of types of the plurality of medical devices includes at least one identifier element that encapsulates at least one identifier associated with at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 4b. The method of any of clauses 1b-3b, wherein the plurality of identifier elements includes at least one identifier element including at least one of the following types of identifier elements: a colored pattern, a reflective pattern, a fluorescent pattern, a predetermined shape and color, a LED pattern, a barcode, a fiducial marker, a hologram marker, or any combination thereof.

Clause 5b. The system of any of clauses 1b-4b, wherein the plurality of identifier elements includes at least one identifier element including a fluorescent coating configured to emit light of a predetermined wavelength, and wherein the one or more image capture devices capture only the light of the predetermined wavelength in the plurality of images.

Clause 6b. The method of any of clauses 1b-5b, wherein the plurality of identifier elements includes at least one identifier element including at least one LED configured to emit light of at least one predetermined wavelength in at least one pattern and/or at least one intensity, and wherein the one or more image capture devices capture only the light of the predetermined wavelength in the plurality of images.

Clause 7b. The method of any of clauses 1b-6b, further comprising: determining, with the one or more processors, based on the at least one pattern and/or the at least one intensity of the emitted light of the at least one predetermined wavelength captured in the plurality of images, a type of a medical device associated with the at least one identifier element.

Clause 8b. The method of any of clauses 1b-7b, wherein the plurality of identifier elements includes at least one identifier element including at least one color changing dye configured to change color over the period of time, and wherein the method further comprises: determining, with the one or more processors, based on a change in the color of the color changing dye in the plurality of images, an amount of time associated with a use of a medical device associated with the at least one identifier element.

Clause 9b. The method of any of clauses 1b-8b, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

Clause 10b. The method of any of clauses 1b-9b, wherein the plurality of identifiers is further associated with a plurality of orientations of the plurality of medical devices, and wherein the method further comprises: determining, with the one or more processors, based on the plurality of identifier elements determined in the plurality of images, the plurality of orientations of the plurality of medical devices within the environment over the period of time; and updating, based on the plurality of orientations of the plurality of medical devices within the environment over the period of time, the database.

Clause 11b. A system comprising: a plurality of identifier elements associated with a plurality of medical devices, wherein the plurality of identifier elements encapsulates a plurality of identifiers associated a plurality of types of the plurality of medical devices; one or more image capture devices configured to capture, over a period of time, a plurality of images of an environment surrounding the one or more image capture devices; and one or more processors programmed and/or configured to: determine, based on the plurality of images, the plurality of identifier elements within the environment over the period of time; determine, based on the plurality of identifier elements determined in the plurality of images, the plurality of types of the plurality of medical devices and a plurality of locations of the plurality of medical devices within the environment over the period of time; and update, based on the plurality of types of the plurality of medical devices and the plurality of locations of the plurality of medical devices within the environment over the period of time, a database including spatial relationships between the plurality of medical devices.

Clause 12b. The system of clause 11b, wherein the plurality of identifiers uniquely identify the plurality of medical devices from each other.

Clause 13b. The system of any of clauses 11b and 12b, wherein the plurality of types of the plurality of medical devices includes at least one identifier element that encapsulates at least one identifier associated with at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 14b. The system of any of clauses 11b-13b wherein the plurality of identifier elements includes at least one identifier element including at least one of the following types of identifier elements: a colored pattern, a reflective pattern, a fluorescent pattern, a predetermined shape and color, a LED pattern, a barcode, a fiducial marker, a hologram marker, or any combination thereof.

Clause 15b. The system of any of clauses 11b-14b, wherein the plurality of identifier elements includes at least one identifier element including a fluorescent coating configured to emit light of a predetermined wavelength, wherein the one or more image capture devices are configured to capture only the light of the predetermined wavelength in the plurality of images.

Clause 16b. The system of any of clauses 11b-15b, wherein the plurality of identifier elements includes at least one identifier element including at least one LED configured to emit light of at least one predetermined wavelength in at least one pattern and/or at least one intensity, wherein the one or more image capture devices are configured to capture only the light of the predetermined wavelength in the plurality of images.

Clause 17b. The system of any of clauses 11b-16b, wherein the one or more processors are further programmed and/or configured to determine, based on the at least one pattern and/or the at least one intensity of the emitted light of the at least one predetermined wavelength captured in the plurality of images, a type of a medical device associated with the at least one identifier element.

Clause 18b. The system of any of clauses 11b-17b, wherein the plurality of identifier elements includes at least one identifier element including at least one color changing dye configured to change color over the period of time, wherein the one or more processors are further programmed and/or configured to determine, based on a change in the color of the color changing dye in the plurality of images, an amount of time associated with a use of a medical device associated with the at least one identifier element.

Clause 19b. The system of any of clauses 11b-18b, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

Clause 20b. The system of any of clauses 11b-19b, wherein the plurality of identifiers is further associated with a plurality of orientations of the plurality of medical devices, and wherein the one or more processors are further programmed and/or configured to: determine, based on the plurality of identifier elements determined in the plurality of images, the plurality of orientations of the plurality of medical devices within the environment over the period of time; and update, based on the plurality of orientations of the plurality of medical devices within the environment over the period of time, the database.

Clause 1c. A method comprising: capturing, with one or more image capture devices, over a period of time, a plurality of images of an environment surrounding the one or more image capture devices; determining, with one or more processors, based on the plurality of images, a plurality of locations of a plurality of medical devices within the environment over the period of time and a plurality of types of the plurality of medical devices; determining, with the one or more processors, based on the plurality of locations of the plurality of medical devices within the environment over the period of time, a plurality of distances between the plurality of medical devices over the period of time; determining, with the one or more processors, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and updating, with the one or more processors, based on the at least one determined event, a database including current connections between the plurality of medical devices.

Clause 2c. The method of clause 1c, wherein the first medical device includes at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 3c. The method of any of clauses 1c and 2c, further comprising: determining, with the one or more processors, based on the plurality of distances between the plurality of medical devices over the period of time and the plurality of types of the plurality of medical devices, one of the following further events: (i) a reuse of the first medical device including a disconnection of the first medical device from the second medical device in the environment and a reconnection of the first medical device to the second medical device in the environment, (ii) a replacement of the first medical device with a new medical device of a same type as the first medical device including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and updating, with the one or more processors, based on the further determined event, the database including the spatial relationships between the plurality of medical devices.

Clause 4c. The method of any of clauses 1c-3c, further comprising: determining, with the one or more processors, based on the plurality of images, a plurality of orientations of the plurality of medical devices within the environment over the period of time, wherein determining the at least one event is further based on the plurality of orientations of the plurality of medical devices within the environment over the period of time.

Clause 5c. The method of any of clauses 1c-4c, wherein the database further includes directions of fluid flows in fluid flow paths through the current connections, wherein the at least one event includes the connection of the first medical device of the plurality of medical devices to the second medical device of the plurality of medical devices, and wherein the method further comprises: determining, with the one or more processors, based on an orientation of the first medical device and an orientation of the second medical device, a direction of a fluid flow in a fluid plow path through the first medical device and the second medical device; and updating, with the one or more processors, based on the direction of the fluid flow in the fluid plow path through the first medical device and the second medical device, the database.

Clause 6c. The method of any of clauses 1c-5c, wherein the first medical device includes at least one of a disinfectant cap and a disinfectant swab such that the connection of the first medical device of the plurality of medical devices to the second medical device of the plurality of medical devices does not form a fluid flow path through the first medical device and the second medical device.

Clause 7c. The method of any of clauses 1c-6c, wherein determining the at least one event further determines a probability associated with the at least one event.

Clause 8c. The method of any of clauses 1c-7c, further comprising: delaying, with the one or more processors, determining the plurality of distances between the plurality of medical devices over the period of time and determining the at least one event until a location of at least one of the first medical device and the second medical device changes in the plurality of images over the period of time.

Clause 9c. The method of any of clauses 1c-8c, further comprising: in response to determining the at least one event, at least one of: providing, with the one or more processors, to a user device, an alert associated with the at least one event; and automatically controlling, with the one or more processors, at least one medical device to stop a flow of fluid in a fluid flow path including the medical device.

Clause 10c. The method of any of clauses 1c-9c, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

Clause 11c. A system comprising: one or more image capture devices configured to capture, over a period of time, a plurality of images of an environment surrounding the one or more image capture devices; one or more processors programmed and/or configured to: determine, based on the plurality of images, a plurality of locations of a plurality of medical devices within the environment over the period of time and a plurality of types of the plurality of medical devices; determine, based on the plurality of locations of the plurality of medical devices within the environment over the period of time, a plurality of distances between the plurality of medical devices over the period of time; determine, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and update, based on the at least one determined event, a database including current connections between the plurality of medical devices.

Clause 12c. The system of clause 11c, wherein the first medical device includes at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 13c. The system of any of causes 11c and 12c, wherein the one or more processors are further programmed and/or configured to: determine, based on the plurality of distances between the plurality of medical devices over the period of time and the plurality of types of the plurality of medical devices, one of the following further events: (i) a reuse of the first medical device including a disconnection of the first medical device from the second medical device in the environment and a reconnection of the first medical device to the second medical device in the environment, (ii) a replacement of the first medical device with a new medical device of a same type as the first medical device including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and update, based on the further determined event, the database including the spatial relationships between the plurality of medical devices.

Clause 14c. The system of any of clauses 11c-13c, wherein the one or more processors are further programmed and/or configured to: determine, based on the plurality of images, a plurality of orientations of the plurality of medical devices within the environment over the period of time, wherein determining the at least one event is further based on the plurality of orientations of the plurality of medical devices within the environment over the period of time.

Clause 15c. The system of any of clauses 11c-14c, wherein the database further includes directions of fluid flows in fluid flow paths through the current connections, wherein the at least one event includes the connection of the first medical device of the plurality of medical devices to the second medical device of the plurality of medical devices, and wherein the one or more processors are further programmed and/or configured to: determine, based on an orientation of the first medical device and an orientation of the second medical device, a direction of a fluid flow in a fluid plow path through the first medical device and the second medical device; and update, based on the direction of the fluid flow in the fluid plow path through the first medical device and the second medical device, the database.

Clause 16c. The system of any of clauses 11c-15c, wherein the first medical device includes at least one of a disinfectant cap and a disinfectant swab such that the connection of the first medical device of the plurality of medical devices to the second medical device of the plurality of medical devices does not form a fluid flow path through the first medical device and the second medical device.

Clause 17c. The system of any of clauses 11c-16c, wherein determining the at least one event further determines a probability associated with the at least one event.

18c. The system of any of clauses 11c-17c, wherein the one or more processors are further programmed and/or configured to: delay determining the plurality of distances between the plurality of medical devices over the period of time and determining the at least one event until a location of at least one of the first medical device and the second medical device changes in the plurality of images over the period of time.

Clause 19c. The system of any of clauses 11c-18c, wherein the one or more processors are further programmed and/or configured to: in response to determining the at least one event, at least one of: provide, to a user device, an alert associated with the at least one event; and automatically control at least one medical device to stop a flow of fluid in a fluid flow path including the medical device.

Clause 20c. The system of any of clauses 11c-19c, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

Clause 1d. A method comprising: capturing, with one or more image capture devices, over a period of time, a plurality of images of an environment surrounding the one or more image capture devices; determining, with one or more processors, based on the plurality of images, a first identifier element associated with a medical device and a second identifier element associated with a glove of a caregiver, wherein the first identifier element encapsulates a first identifier associated with the medical device, and wherein the second identifier element encapsulates a second identifier associated with the glove of the caregiver; determining, with the one or more processors, based on the first identifier element in the plurality of images, the medical device and a location of the medical device within the environment over the period of time; determining, with the one or more processors, based on the second identifier element in the plurality of images, the glove of the caregiver and a location of the glove of the caregiver within the environment over the period of time; determining, with the one or more processors, based on the location of the medical device within the environment over the period of time and the location of the glove of the caregiver within the environment over the period of time and the location, at least one event associated with the medical device; and updating, with the one or more processors, based on the at least one determined event, a database including events associated with the environment.

Clause 2d. The method of clause 1d, further comprising: determining, with the one or more processors, based on the plurality of images, a location of a further medical device within the environment over the period of time and a type of the further medical device; and determining, with the one or more processors, based on the location of the further medical device over the period of time and at least one of (i) the location of medical device within the environment over the period of time and (ii) the location of the glove of the caregiver within the environment over the period of time, a linear distance change and an angular distance change between the further medical device and the at least one of the medical device and the glove of the caregiver over the period of time, wherein determining the at least one event is further based on the linear distance change and the angular distance change between the medical device and the glove of the caregiver.

Clause 3d. The method of any of clauses 1d and 2d, wherein the at least one event includes a scrubbing or disinfecting event.

Clause 4d. The method of any of clauses 1d-3d, further comprising: determining, with the one or more processors, based on the on the linear distance change and the angular distance change between the medical device and the glove of the caregiver over the period of time, a duration of the scrubbing action.

Clause 5d. The method of any of clauses 1d-4d, wherein the further medical device includes a disinfectant swab or wipe.

Clause 6d. The method of any of clauses 1d-5d, wherein the medical device includes a needless connector.

Clause 7d. The method of any of clauses 1d-6d, wherein the at least one event includes a catheter dressing change event, and wherein the medical device includes a catheter dressing.

Clause 8d. The method of any of clauses 1d-7d, wherein the second identifier element includes a predetermined color of the glove of the caregiver.

Clause 9d. The method of any of clauses 1d-8d, wherein the first identifier element includes at least one of: a colored pattern, a reflective pattern, a fluorescent pattern, a LED pattern, a barcode, a fiducial marker, a hologram marker, or any combination thereof.

Clause 10d. The method of any of clauses 1d-9d, further comprising: in response to determining the at least one event, at least one of: providing, with the one or more processors, to a user device, an alert associated with the at least one event; and automatically controlling, with the one or more processors, at least one medical device to stop a flow of fluid in a fluid flow path including the medical device.

Clause 11d. A system comprising: a first identifier element associated with a medical device, wherein the first identifier element encapsulates a first identifier associated with the medical device; a second identifier element associated with a glove of a caregiver, wherein the second identifier element encapsulates a second identifier associated with the glove of the caregiver; one or more image capture devices configured to capture, over a period of time, a plurality of images of an environment surrounding the one or more image capture devices; and one or more processors programmed and/or configured to: determine, based on the plurality of images, the first identifier element associated with the medical device and the second identifier element associated with the glove of a caregiver; determine, based on the first identifier element in the plurality of images, the medical device and a location of the medical device within the environment over the period of time; determine, based on the second identifier element in the plurality of images, the glove of the caregiver and a location of the glove of the caregiver within the environment over the period of time; determine, based on the location of the medical device within the environment over the period of time and the location of the glove of the caregiver within the environment over the period of time and the location, at least one event associated with the medical device; and update, based on the at least one determined event, a database including events associated with the environment.

Clause 12d. The system of clause 11d, wherein the one or more processors are further programmed and/or configured to: determine, based on the plurality of images, a location of a further medical device within the environment over the period of time and a type of the further medical device; and determine, based on the location of the further medical device over the period of time and at least one of (i) the location of medical device within the environment over the period of time and (ii) the location of the glove of the caregiver within the environment over the period of time, a linear distance change and an angular distance change between the further medical device and the at least one of the medical device and the glove of the caregiver over the period of time, wherein determining the at least one event is further based on the linear distance change and the angular distance change between the medical device and the glove of the caregiver.

Clause 13d. The system of any of clauses 11d and 12d, wherein the at least one event includes a scrubbing or disinfecting event.

Clause 14d. The system of any of clauses 11d-13d, wherein the one or more processors are further programmed and/or configured to: determine, based on the on the linear distance change and the angular distance change between the medical device and the glove of the caregiver over the period of time, a duration of the scrubbing action.

Clause 15d. The system of any of clauses 11d-14d, wherein the further medical device includes a disinfectant swab or wipe.

Clause 16d. The system of any of clauses 11d-15d, wherein the medical device includes a needless connector.

Clause 17d. The system of any of clauses 11d-16d, wherein the at least one event includes a catheter dressing change event, and wherein the medical device includes a catheter dressing.

Clause 18d. The system of any of clauses 11d-17d, wherein the second identifier element includes a predetermined color of the glove of the caregiver.

Clause 19d. The system of any of clauses 11d-18d, wherein the first identifier element includes at least one of: a colored pattern, a reflective pattern, a fluorescent pattern, a LED pattern, a barcode, a fiducial marker, a hologram marker, or any combination thereof.

Clause 20d. The system of any of clauses 11d-19d, wherein the one or more processors are further programmed and/or configured to: in response to determining the at least one event, at least one of: provide, to a user device, an alert associated with the at least one event; and automatically control, at least one medical device to stop a flow of fluid in a fluid flow path including the medical device.

Clause 1e. A method comprising: capturing, with one or more image capture devices, over a period of time, a plurality of images of an environment surrounding the one or more image capture devices; determining, with one or more processors, based on the plurality of images, a location of a plunger of a syringe relative to a barrel of the syringe in the environment over the period of time; determining, with the one or more processors, based on the location of the plunger of the syringe relative to the barrel of the syringe over the period of time, at least one fluid delivery from the syringe; and updating, with the one or more processors, based on the at least one determined fluid delivery, a database including fluid deliveries associated with the environment.

Clause 2e. The method of clause 1e, wherein determining the least one fluid delivery further includes determining, based on the location of the plunger of the syringe relative to the barrel of the syringe over the period of time, at least one of an amount of fluid delivered by the at least one fluid delivery and a fluid flow rate of the at least one fluid delivery.

Clause 3e. The method of any of clauses 1e and 2e, further comprising: determining, with the one or more processors, based on the fluid flow rate of the at least one fluid delivery, a type of the fluid associated with the at least one fluid delivery.

Clause 4e. The method of any of clauses 1e-3e, further comprising: determining, with the one or more processors, based on the plurality of images, a location of a glove of a caregiver within the environment over the period of time; and determining, with the one or more processors, based on the location of the glove of the caregiver within the environment over the period of time, a flush technique associated with the at least one fluid delivery, wherein the flush technique includes a pulsatile flush or a continuous flush.

Clause 5e. The method of any of clauses 1e-4e, wherein determining the location of the plunger of the syringe relative to the barrel of the syringe over the period of time further includes determining, based on the plurality of images, a first identifier element associated with the plunger of the syringe and a second identifier element associated with the barrel of the syringe, wherein the location of the plunger of a syringe relative to a barrel of the syringe in the environment over the period of time is determined based on the first identifier element in the plurality of images and the second identifier element in the plurality of images.

Clause 6e. The method of any of clauses 1e-5e, wherein determining the location of the plunger of the syringe relative to the barrel of the syringe in the environment over the period of time includes determining, based on the first identifier element and the second identifier element in the plurality of images, a distance between the first identifier element and the second identifier element as a distance between the plunger and the barrel of the syringe.

Clause 7e. The method of any of clauses 1e-6e, wherein the first identifier element and the second identifier element include at least one of the following: a colored pattern, a reflective pattern, a fluorescent pattern, a predetermined shape and color, a LED pattern, a barcode, a fiducial marker, a hologram marker, or any combination thereof.

Clause 8e. The method of any of clauses 1e-7e, further comprising: determining, with the one or more processors, based on the plurality of images, a color of a fluid contained in the syringe; and determining, with the one or more processors, based on the color of the fluid, a type of the fluid associated with the at least one fluid delivery.

Clause 9e. The method of any of clauses 1e-8e, wherein the plurality of images include at least one infrared (IR) image, and wherein the method further comprises: determining, with the one or more processors, based on the at least one IR image, a temperature of a fluid contained in the syringe; and determining, with the one or more processors, based on the temperature of the fluid contained in the syringe, a type of the fluid associated with the at least one fluid delivery.

Clause 10e. The method of any of clauses 1e-9e, wherein the plurality of images include at least one infrared (IR) image, and wherein the method further comprises: determining, with the one or more processors, based on the at least one IR image, a temperature of a fluid contained in the syringe; comparing, with the one or more processors, the temperature of the fluid contained in the syringe to a threshold temperature associated with the fluid; and in response to determining that the temperature of the fluid contained in the syringe satisfies the threshold temperature, automatically controlling, with the one or more processors, at least one medical device to stop the at least one fluid delivery from the syringe.

Clause 11e. A system comprising: one or more image capture devices configured to capture, over a period of time, a plurality of images of an environment surrounding the one or more image capture devices; and one or more processors programmed and/or configured to: determine, based on the plurality of images, a location of a plunger of a syringe relative to a barrel of the syringe in the environment over the period of time; determine, based on the location of the plunger of the syringe relative to the barrel of the syringe over the period of time, at least one fluid delivery from the syringe; and update, based on the at least one determined fluid delivery, a database including fluid deliveries associated with the environment.

Clause 12e. The system of clause 11e, wherein the one or more processors are further programmed and/or configured to: determine, based on the location of the plunger of the syringe relative to the barrel of the syringe over the period of time, at least one of an amount of fluid delivered by the at least one fluid delivery and a fluid flow rate of the at least one fluid delivery.

Clause 13e. The system of any of clauses 11e and 12e, wherein the one or more processors are further programmed and/or configured to: determine, based on the fluid flow rate of the at least one fluid delivery, a type of the fluid associated with the at least one fluid delivery.

Clause 14e. The system of any of clauses 11e-13e, wherein the one or more processors are further programmed and/or configured to: determine, based on the plurality of images, a location of a glove of a caregiver within the environment over the period of time; and determine, based on the location of the glove of the caregiver within the environment over the period of time, a flush technique associated with the at least one fluid delivery, wherein the flush technique includes a pulsatile flush or a continuous flush.

Clause 15e. The system of any of clauses 11e-14e, wherein the one or more processors are programmed and/or configured to determine the location of the plunger of the syringe relative to the barrel of the syringe over the period of time by determining, based on the plurality of images, a first identifier element associated with the plunger of the syringe and a second identifier element associated with the barrel of the syringe, wherein the location of the plunger of a syringe relative to a barrel of the syringe in the environment over the period of time is determined based on the first identifier element in the plurality of images and the second identifier element in the plurality of images.

Clause 16e. The system of any of clauses 11e-15e, wherein the one or more processors are programmed and/or configured to determine the location of the plunger of the syringe relative to the barrel of the syringe in the environment over the period of time by determining, based on the first identifier element and the second identifier element in the plurality of images, a distance between the first identifier element and the second identifier element as a distance between the plunger and the barrel of the syringe.

Clause 17e. The system of any of clauses 11e-16e, wherein the first identifier element and the second identifier element include at least one of the following: a colored pattern, a reflective pattern, a fluorescent pattern, a predetermined shape and color, a LED pattern, a barcode, a fiducial marker, a hologram marker, or any combination thereof.

Clause 18e. The system of any of clauses 11e-17e, wherein the one or more processors are further programmed and/or configured to: determine, based on the plurality of images, a color of a fluid contained in the syringe; and determine, based on the color of the fluid, a type of the fluid associated with the at least one fluid delivery.

Clause 19e. The system of any of clauses 11e-18e, wherein the plurality of images include at least one infrared (IR) image, and wherein the one or more processors are further programmed and/or configured to: determine, based on the at least one IR image, a temperature of a fluid contained in the syringe; and determine, based on the temperature of the fluid contained in the syringe, a type of the fluid associated with the at least one fluid delivery.

Clause 20e. The system of any of clauses 11e-19e, wherein the plurality of images include at least one infrared (IR) image, and wherein the one or more processors are further programmed and/or configured to: determine, based on the at least one IR image, a temperature of a fluid contained in the syringe; compare, the temperature of the fluid contained in the syringe to a threshold temperature associated with the fluid; and in response to determining that the temperature of the fluid contained in the syringe satisfies the threshold temperature, automatically control, at least one medical device to stop the at least one fluid delivery from the syringe.

Clause 1f. A method comprising: capturing, with one or more image capture devices, over a period of time, a plurality of images of an environment surrounding the one or more image capture devices; determining, with one or more processors, based on the plurality of images, a state of a package containing a medical device over the period of time; determining, with the one or more processors, based on the state of the package over the period of time, whether the medical device is removed from the package; and updating, with the one or more processors, based on a determination that the medical device is removed from the package, a database including medical devices in the environment.

Clause 2f. The method of clause 1f, wherein the package includes a removable first layer covering a second layer, wherein the first layer including a first color, wherein the second layer includes a second color different than the first color, and wherein removal of the first layer from the package reveals the second layer.

Clause 3f. The method of any of clauses 1f and 2f, wherein the first layer is at least partially transparent.

Clause 4f. The method of any of clauses 1f-3f, wherein a color of the package is configured to change when exposed to air.

Clause 5f. The method of any of clauses 1f-4f, wherein a portion of the package is transparent such that the medical device contained within the package is visible through the transparent portion of the package.

Clause 6f. The method of any of clauses 1f-5f, wherein the package is associated with a first identifier element, wherein the medical device is associated with a second identifier element different than the first identifier element, and wherein the state of the package is determined based on a location of the first identifier element with respect to a location of the second identifier element.

Clause 7f. The method of any of clauses 1f-6f, further comprising: determining, based on the plurality of images, a location of a glove of a caregiver within the environment over the period of time with respect to a location of the package, wherein the state of the package is determined based on the location of the glove of the caregiver within the environment over the period of time with respect to the location of the package.

Clause 8f. The method of any of clauses 1f-7f, wherein the package includes a removable first layer covering a second layer, wherein the removable first layer includes a first identifier element, wherein the second layer includes a second identifier element, wherein the removable first layer is at least partially transparent, and wherein determining the state of the package further includes: determining, based on the plurality of images, a distance between the first identifier element and the second identifier element; and determining, based on the distance between the first identifier element and the second identifier element, whether the package is faulty; and in response to determining that the package is faulty, providing, to a user device, an alert associated with the faulty package.

Clause 9f. The method of any of clauses 1f-8f, further comprising: determining, with the one or more processors, based on the plurality of images, a plurality of locations of a plurality of medical devices within the environment over the period of time and a plurality of types of the plurality of medical devices; and determining, with the one or more processors, based on the plurality of locations of the plurality of medical devices within the environment over the period of time, the plurality of types of the plurality of medical devices, and the state of the package over the period of time, at least one event of the following events: (i) a reuse of the medical device including a connection of the medical device to two or more medical devices in the environment over the period of time, (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device.

Clause 10f. The method of any of clauses 1f-9f, wherein the medical device includes a disinfectant swab or wipe.

Clause 11f. A system comprising: a package containing a medical device; one or more image capture devices configured to capture, over a period of time, a plurality of images of an environment surrounding the one or more image capture devices; and one or more processors programmed and/or configured to: determine, based on the plurality of images, a state of the package over the period of time; determine, based on the state of the package over the period of time, whether the medical device is removed from the package; and update, based on a determination that the medical device is removed from the package, a database including medical devices in the environment.

Clause 12f. The system of clause 11f, wherein the package includes a removable first layer covering a second layer, wherein the first layer including a first color, wherein the second layer includes a second color different than the first color, and wherein removal of the first layer from the package reveals the second layer.

Clause 13f. The system of any of clauses 11f and 12f, wherein the first layer is at least partially transparent.

Clause 14f. The system of any of clauses 11f-13f, wherein a color of the package is configured to change when exposed to air.

Clause 15f. The system of any of clauses 11f-14f, wherein a portion of the package is transparent such that the medical device contained within the package is visible through the transparent portion of the package.

Clause 16f. The system of any of clauses 11f-15f, wherein the package is associated with a first identifier element, wherein the medical device is associated with a second identifier element different than the first identifier element, and wherein the state of the package is determined based on a location of the first identifier element with respect to a location of the second identifier element.

Clause 17f. The system of any of clauses 11f-16f, wherein the one or more processors are further programmed and/or configured to determine, based on the plurality of images, a location of a glove of a caregiver within the environment over the period of time with respect to a location of the package, wherein the state of the package is determined based on the location of the glove of the caregiver within the environment over the period of time with respect to the location of the package.

Clause 18f. The system of any of clauses 11f-17f, wherein the package includes a removable first layer covering a second layer, wherein the removable first layer includes a first identifier element, wherein the second layer includes a second identifier element, wherein the removable first layer is at least partially transparent, and wherein the one or more processors are further programmed and/or configured to: determine, based on the plurality of images, a distance between the first identifier element and the second identifier element; and determine, based on the distance between the first identifier element and the second identifier element, whether the package is faulty; and in response to determining that the package is faulty, provide, to a user device, an alert associated with the faulty package.

Clause 19f. The system of any of clauses 11f-18f, wherein the one or more processors are further programmed and/or configured to: determine, based on the plurality of images, a plurality of locations of a plurality of medical devices within the environment over the period of time and a plurality of types of the plurality of medical devices; and determine, based on the plurality of locations of the plurality of medical devices within the environment over the period of time, the plurality of types of the plurality of medical devices, and the state of the package over the period of time, at least one event of the following events: (i) a reuse of the medical device including a connection of the medical device to two or more medical devices in the environment over the period of time, (ii) a replacement of the medical device with a new medical device of a same type as the medical device in the environment, and (iii) a replacement of the medical device with different medical device of a different type than the medical device in the environment.

Clause 20f. The system of any of clauses 11f-19f, wherein the medical device includes a disinfectant swab or wipe.

Clause 1g. A method comprising: capturing, with one or more image capture devices, over a period of time, a plurality of images of an environment surrounding the one or more image capture devices; obtaining, with one or more processors, from a data source other than the one or more image capture devices, auxiliary data associated with at least one of: at least one image of the environment during the period of time and audio recorded in the environment during the period of time; determining, with the one or more processors, based on the plurality of images and the auxiliary data, a location of a medical device within the environment over the period of time; and updating, with the one or more processors, based on the location of the medical device within the environment over the period of time, a database including a spatial relationship of the medical device and at least one other medical device.

Clause 2g. The method of clause 1g, wherein the medical device is not visible in at least a portion of the plurality of images.

Clause 3g. The method of any of clauses 1g and 2g, wherein the medical device includes a catheter, and wherein the auxiliary data includes at least one of: a tubing type of the catheter, a size of the catheter, a shape of the catheter, and a location of a catheter insertion site of the catheter on a patient.

Clause 4g. The method of any of clauses 1g-3g, wherein the auxiliary data is associated with the audio recorded in the environment during the period of time.

Clause 5g. The method of any of clauses 1g-4g, wherein the audio includes a predetermined signal associated with the medical device.

Clause 6g. The method of any of clauses 1g-5g, wherein the medical device includes an infusion pump.

Clause 7g. The method of any of clauses 1g-6g, further comprising: determining, with the one or more processors, based on the plurality of images and the auxiliary data, a plurality of locations of a plurality of medical devices within the environment over the period of time and a plurality of types of the plurality of medical devices, wherein the plurality of medical devices includes the medical device and the at least one other medical device, wherein the medical device and the at least one other medical device are configured to emit a predetermined audible signal when connected; and determining, with the one or more processors, based on the plurality of locations of the plurality of medical devices within the environment over the period of time and the plurality of types of the plurality of medical devices, at least one connection between two or more medical devices of the plurality of medical devices; and updating, with the one or more processors, based on the at least one connection determined between the two or more medical devices, the database.

Clause 8g. The method of any of clauses 1g-7g, wherein the audio includes the predetermined audible signal associated with connection of the medical device and the at least one other medical device.

Clause 9g. The method of any of clauses 1g-8g, wherein the medical device includes at least one of: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 10g. The method of any of clauses 1g-9g, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LIDAR sensor, or any combination thereof.

Clause 11g. A system comprising: one or more image capture devices configured to capture, over a period of time, a plurality of images of an environment surrounding the one or more image capture devices; and one or more processors programmed and/or configured to: obtain, from a data source other than the one or more image capture devices, auxiliary data associated with at least one of: at least one image of the environment during the period of time and audio recorded in the environment during the period of time; determine, based on the plurality of images and the auxiliary data, a location of a medical device within the environment over the period of time; and update, based on the location of the medical device within the environment over the period of time, a database including a spatial relationship of the medical device and at least one other medical device.

Clause 12g. The system of clause 11g, wherein the medical device is not visible in at least a portion of the plurality of images.

Clause 13g. The system of any of clauses 11g and 12g, wherein the medical device includes a catheter, and wherein the auxiliary data includes at least one of: a tubing type of the catheter, a size of the catheter, a shape of the catheter, and a location of a catheter insertion site of the catheter on a patient.

Clause 14g. The system of any of clauses 11g-13g, wherein the auxiliary data is associated with the audio recorded in the environment during the period of time.

Clause 15g. The system of any of clauses 11g-14g, wherein the audio includes a predetermined signal associated with the medical device.

Clause 16g. The system of any of clauses 11g-15g, wherein the medical device includes an infusion pump.

Clause 17g. The system of any of clauses 11g-16g, wherein the one or more processors are further programmed and/or configured to: determine, based on the plurality of images and the auxiliary data, a plurality of locations of a plurality of medical devices within the environment over the period of time and a plurality of types of the plurality of medical devices, wherein the plurality of medical devices includes the medical device and the at least one other medical device, wherein the medical device and the at least one other medical device are configured to emit a predetermined audible signal when connected; and determine, based on the plurality of locations of the plurality of medical devices within the environment over the period of time and the plurality of types of the plurality of medical devices, at least one connection between two or more medical devices of the plurality of medical devices; and update, based on the at least one connection determined between the two or more medical devices, the database.

Clause 18g. The system of any of clauses 11g-17g, wherein the audio includes the predetermined audible signal associated with connection of the medical device and the at least one other medical device.

Clause 19g. The system of any of clauses 11g-18g, wherein the medical device includes at least one of: a peripheral IV catheter (PIVC), a peripherally inserted central catheter (PICC), a midline catheter, a central venous catheter (CVC), a needleless connector, a catheter dressing, a disinfectant cap, a disinfectant swab, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Clause 20g. The system of any of clauses 11g-19g, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

### BRIEF DESCRIPTION OF THE DRAWINGS

Additional advantages and details are explained in greater detail below with reference to the exemplary embodiments that are illustrated in the accompanying schematic figures, in which:

FIG. 1A is a diagram of non-limiting embodiments or aspects of an environment in which systems, devices, products, apparatus, and/or methods, described herein, can be implemented;

FIG. 1B is a diagram of non-limiting embodiments or aspects of an implementation of an environment in which systems, devices, products, apparatus, and/or methods, described herein, can be implemented;

FIG. 2 is a diagram of non-limiting embodiments or aspects of components of one or more devices and/or one or more systems of FIGS. 1A and 1B;

FIG. 3 is a flowchart of non-limiting embodiments or aspects of a process for vascular access management;

FIG. 4 is a flowchart of non-limiting embodiments or aspects of a process for vascular access management;

FIG. 5 is a flowchart of non-limiting embodiments or aspects of a process for vascular access management;

FIG. 6 is a flowchart of non-limiting embodiments or aspects of a process for vascular access management;

FIGS. 7A and 7B are a flowchart of non-limiting embodiments or aspects of a process for vascular access management;

FIG. 8 is a flowchart of non-limiting embodiments or aspects of a process for vascular access management;

FIG. 9 is a flowchart of non-limiting embodiments or aspects of a process for vascular access management;

FIG. 10 is a perspective view of non-limiting embodiments or aspects of example implementations of a medical device;

FIG. 11 is a perspective view of non-limiting embodiments or aspects of implementations of identifier elements;

FIG. 12 is a perspective view of non-limiting embodiments or aspects of implementations of identifier elements;

FIG. 13 illustrates an example visual representation of the implementation of the environment of FIG. 1B;

FIG. 14 is a perspective view of non-limiting embodiments or aspects of a scrubbing event; and

FIG. 15 is a perspective view of non-limiting embodiments or aspects of a syringe including a first identifier element associated with a plunger of the syringe and a second identifier element associated with a barrel of the syringe.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices directly or indirectly communicating in the network environment may constitute a “system” or a “computing system”.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

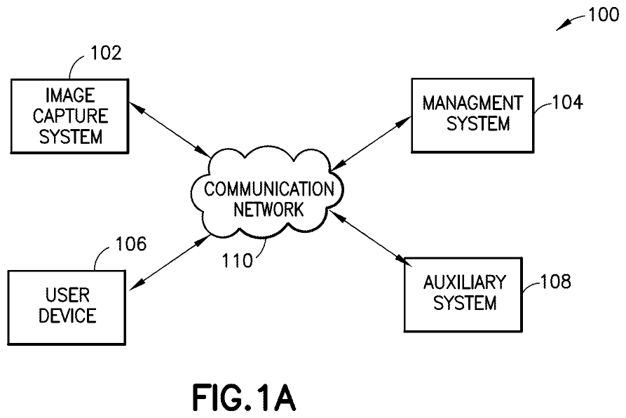

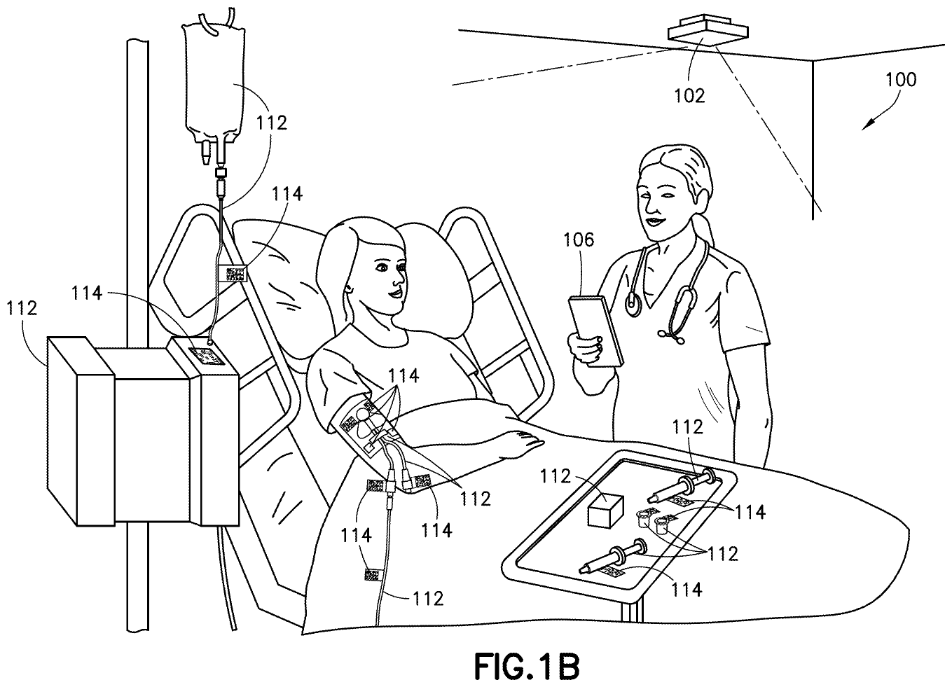

Referring now to FIG. 1A, FIG. 1A is a diagram of an example environment 100 in which devices, systems, methods, and/or products described herein, may be implemented. As shown in FIG. 1A, environment 100 includes image capture system 102, management system 104, user device 106, auxiliary system 108, and/or communication network 110. Systems and/or devices of environment 100 can interconnect via wired connections, wireless connections, or a combination of wired and wireless connections.

Referring also to FIG. 1B, FIG. 1B is a diagram of non-limiting embodiments or aspects of an implementation of environment 100 in which systems, devices, products, apparatus, and/or methods, described herein, can be implemented. For example, as shown in FIG. 1B, environment 100 may include a hospital room including a patient, one or more medical devices 112, one or more identifier elements 114 associated with the one or more medical devices 112, and/or a caretaker (e.g., a nurse, etc.).

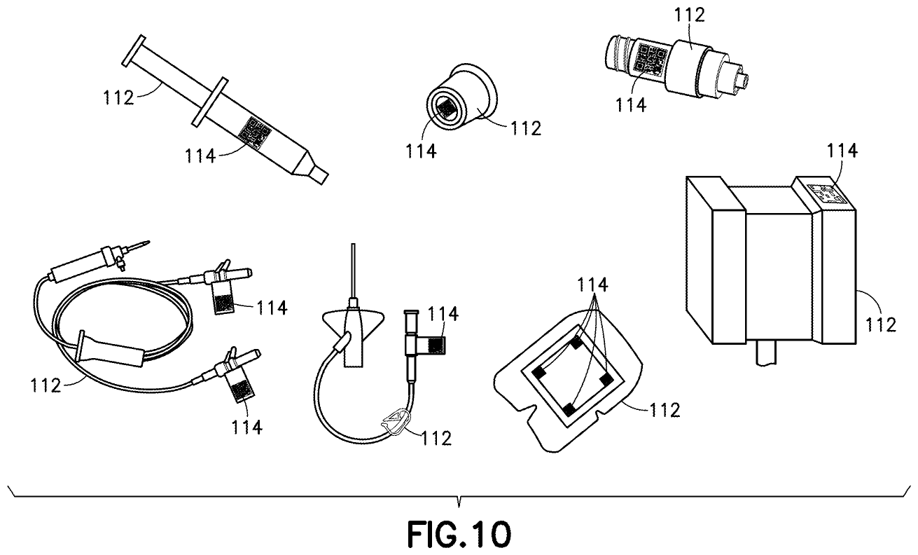

A medical device 112 may enter environment 100 (e.g., via the caretaker, etc.), remain in environment 100 for a period of time (or indefinitely) during which the medical device 112 may move within environment 100 and/or interact with (e.g., connect to, disconnect from, etc.) one or more other medical devices 112, the patient, and/or the caretaker, and/or exit environment 100 at a subsequent time after entering environment 100 (e.g., via the caretaker, etc.). A medical device 112 may include a disposable medical device or a reusable medical device. For example, a medical device 112 may include at least one of the following types of medical devices a peripheral IV catheter (PIVC), a peripherally inserted central catheter (PICC), a midline catheter, a central venous catheter (CVC), a needleless connector, a catheter dressing, a catheter stabilization device, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof. FIG. 10 provides a perspective view of non-limiting embodiments or aspects of implementations of a medical device 112. As described in more detail herein below, in some non-limiting embodiments or aspects, image capture system 102 and/or management system 104 may detect and use a shape, a size, a movement or trajectory, a location, and/or an orientation (e.g., relative to a camera, relative to a patient, relative to a feature of the environment, such as a floor, a bed, a calibration marker, and/or the like, relative to another medical device, etc.) of a medical device 112 to identify a type of medical device 112 and/or to uniquely identify medical device 112 from other medical devices in environment 100, as well as to track locations of the medical device 112 in environment 100 and/or determine events associated with the medical device 112.

Detection of a shape, a size, a movement or trajectory, a location, an orientation, and/or the like of an object may be computationally expensive and/or error-prone. For example, a camera based object detection system may make a mistake in identifying similar objects and/or miss (e.g., fail to detect, etc.) an object in a noisy environment. Accordingly, in some non-limiting embodiments or aspects, an identifier element 114 (e.g., a tag, a label, a code, etc.) may be associated with (e.g., removably attached to, permanently attached to, integrated with, implemented on, etc.) a medical device 112. In some non-limiting embodiments or aspects, each medical device 112 in environment 100 may be associated with an identifier element 114. In some non-limiting embodiments or aspects, only a portion the medical devices 112 in environment 100 may be associated with identifier elements 114. In some non-limiting embodiments or aspects, none of the medical devices 112 in environment 100 may be associated with identifier elements 114. As described in more detail herein below, in some non-limiting embodiments or aspects, image capture system 102 and/or management system 104 may detect and use a shape, a size, a movement or trajectory, a location, and/or an orientation of an identifier element to identify a type of medical device 112 associated with the identifier element 114 and/or to uniquely identify medical device 112 associated with the identifier element 114 from other medical devices in environment 100, as well as to track locations of the medical device 112 associated with the identifier element 114 in environment 100 and/or determine events associated with the medical device 112 associated with the identifier element 114.

An identifier element 114 may encapsulate an identifier associated with a type of a medical device 112 associated with the identifier element 114 and/or uniquely identify the medical device 112 associated with the identifier element 114 from other medical devices and/or indicate an orientation of the medical device 112 within environment 100 and/or with respect to another medical device 112 (e.g., a fluid flow path direction through a medical device 1112, an input or inlet position and an output or outlet position of a medical device, etc.). For example, an identifier element 114 may encapsulate an identifier associated with at least one of the following types of medical devices: a peripheral IV catheter (PIVC), a peripherally inserted central catheter (PICC), a midline catheter, a central venous catheter (CVC), a needleless connector, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof, and/or uniquely identify a medical device 112 from other medical devices including identifiers associated with a same type of medical device. In such an example, an identifier element 114 may include at least one of the following: a colored pattern, a reflective pattern, a fluorescent pattern, a predetermined shape and color, a LED pattern, a barcode (e.g., a 1D barcode, a 2D barcode, etc.), a fiducial marker, a hologram marker, or any combination thereof, which may encapsulate the identifier.

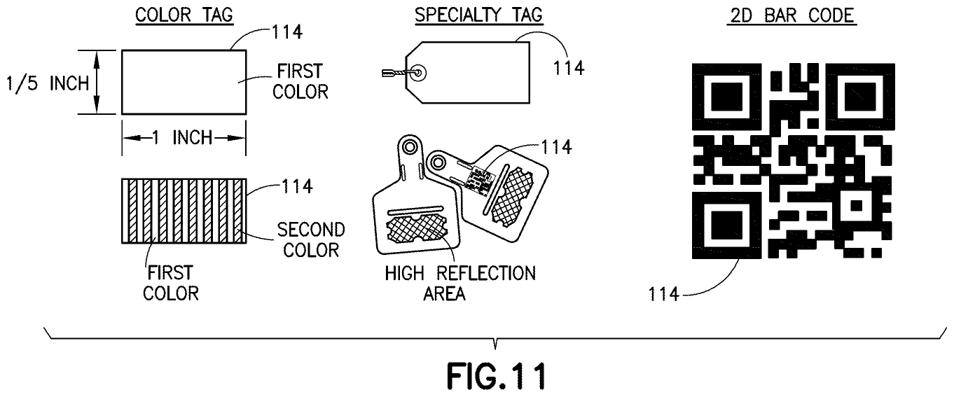

In some non-limiting embodiments or aspects, an identifier element 114 may include one or more colored areas, one or more reflective areas, one or more fluorescent areas, or any combination thereof that encapsulate an identifier. For example, an identifier element 114 may include one or more high-reflection areas such as, mirror surface particles, corner or edge reflectors, and/or the like, that encapsulate an identifier and render the identifier element 114 brighter than ambient illumination in environment 100. In such an example, an identifier element 114 may include a fluorescent coating or pattern on a medical device 112 that encapsulates an identifier by emitting light of a predetermined wavelength detectable in infrared by an image capture device including a filter configured to filter non-infrared light. In such an example, an identifier element 114 may include a tag or label having a predetermined shape and/or a predetermined color and/or color pattern that encapsulates an identifier (e.g., a green tag in a shape of a star, a red tag in a shape of a square, etc.). For example, an identifier element 114 may include a unique geometry and/or shape to differentiate itself from other identifier elements 114, and/or bars that wrap around cylindrical objects, grids, and/or patterns of shapes may be included in identifier element 114 for further identification and differentiation from other identifier elements 114. FIG. 11 is a perspective view of non-limiting embodiments or aspects of implementations of identifier elements 114 including color tags having a predetermined shape (e.g., a rectangle 0.5 inches by 1 inches, etc.) and/or a predetermined color (e.g., a first color, red, etc.) and/or color pattern (e.g., a first color and a second color in a pattern, red and blue in a pattern, etc.), fluorescent and/or reflective tags, and/or bar codes.

In some non-limiting embodiments or aspects, an identifier element 114 may include colors selected (e.g., optimized, etc.) to be detected by image capture system 102. For example, for image capture system 102 may include an RGB camera, and an identifier element 114 may include variable color regions to create unique tag identities. As an example, an individual color used in a variable color region may be created from a percentage (e.g., 0%, 50% or 100%) of one of R, G, and B, and R, G, and B can be used to create 33 or 27 color combinations of variable color regions for reliable differentiation of color. In such an example, multiple variable colors can be placed adjacent to one another to create even more combinations, such as a 2×2 grid of colors, 3 parallel bars of color, and/or the like.

In some non-limiting embodiments or aspects, an identifier element 114 may include color calibration areas positioned adjacent to variable color regions to calibrate color in a wider range of lighting conditions. For example, for a 2×2 grid, a cell (1,1) in an upper-left corner of the gird may include a predetermined and/or standard calibration color region (e.g., neutral gray, etc.), and image capture system 102 and/or management system 104 may use the predetermined and/or standard calibration color region to calibrate colors in images used to detect or determine the identifier element 114 in those images. In such an example, image capture system 102 and/or management system 104 may use the predetermined and/or standard calibration color region to orient identifier element 114 to determine how to properly rotate and decode the colors in identifier element 114 to decode the identifier encapsulated by the identifier element 114 and/or track the identifier element 114 within environment 100.

Accordingly, non-limiting embodiments or aspects of the present disclosure may use unique tags that identify medical devices individually and/or as a category or type of medical device for more robust image segmentation input, which may avoid the use of more standard bar code technology that may be more difficult to resolve without higher-cost cameras by instead using lower spatial resolution images, and lower cost cameras and processing. Further, in some non-limiting embodiments or aspects, additionally or alternatively to variable identifier elements 114 that identify product categories (e.g., by SKU, etc.), identifier elements 14 may include a hyper-variable region in which random hyper-variable tags may be applied during manufacturing. For example, if there are a predetermined number of unique identifiers (e.g., one hundred unique random identifiers, etc.), for any given patient, medical devices 112 may be uniquely identified, even if the medical devices 112 have the same SKU.

In some non-limiting embodiments or aspects, an identifier element 114 may include at least one light emitting diode (LED) (e.g., an RGB LED, an IR LED, etc.) configured to emit light of at least one predetermined wavelength in at least one predetermined pattern (e.g., a color code, a dynamic pattern, etc.) and/or at least one predetermined intensity, which encapsulates an identifier. For example, an identifier element 114 may include a battery (e.g., a rechargeable battery, a single use battery, a replaceable battery, etc.), an energy harvester (e.g., a thermoelectric energy harvester, a photovoltaic energy harvester, a piezoelectric energy harvester, etc.), a wireless power receiver (e.g., an RFID device, etc.), or any combination thereof that is configured to power the at least one LED and/or a controller configured to control the at least one LED to emit the light encapsulating the identifier, and management system 104 may analyze the light captured in images to decode the identifier encapsulated by the identifier element 114 and/or track the identifier element 114 within environment 100.

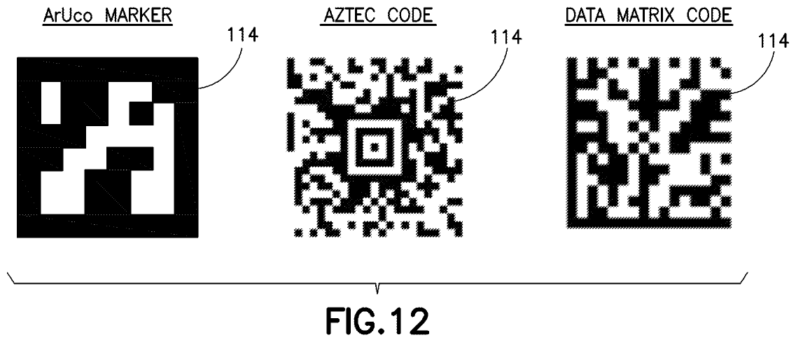

In some non-limiting embodiments or aspects, an identifier element 114 may include a 1D barcode and/or a 2D barcode (e.g., a QR code, an Aztec code, a Data Matrix code, an ArUco marker, etc.) that encapsulates an identifier. For example, as described in more detail herein below, in some non-limiting embodiments or aspects, image capture system 102 and/or management system 104 may detect and/or track an identifier element 114 within environment 100 by detecting three square boxes on QR codes to reposition or orient an image and read a pattern in the QR code to identify a type of medical device 112 associated with the identifier element 114. For example, FIG. 12 illustrates non-limiting embodiments or aspects of implementations of identifier elements 114 including an ArUco marker, an Aztec code, and a Data Matrix code.

In some non-limiting embodiments or aspects, an identifier element 114 may include at least one color changing dye configured to change color over a period of time. For example, as described in more detail herein below, in some non-limiting embodiments or aspects, image capture system 102 and/or management system 104 may determine, based on a change in the color of the color changing dye in a plurality of images, an amount of time associated with a use of a medical device 112 associated with the identifier element 114 (e.g., an amount of time since the medical device 112 is removed from a package, an amount of time medical device 112 is in environment 100, etc.).

Image capture system 102 (e.g., a camera system, a sensor system, etc.) may include one or more image capture devices (e.g., one or more cameras, one or more sensors, etc.) configured to capture, over a period of time, a plurality of images (e.g., image data, a series of images, a video, a live or real-time video feed, etc.) of an environment (e.g., environment 100, etc.) surrounding the one or more image capture devices. For example, an image capture device may include at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.

In some non-limiting embodiments or aspects, image capture system 102 may include a single camera configured to detect or capture only identifier elements 114 (e.g., from background, from other objects in environment 100, etc.). For example, image capture system may include a single wide lens camera in user device 106, such as a tablet computer, and/or the like, that includes built-in auto-focus and image stabilization features. In some non-limiting embodiments or aspects, image capture system 102 may include a plurality of cameras configured to generate images with depth and/or to capture images from multiple different angles or fields-of-view to resolve occlusion of an object in a field of view of a single camera. In some non-limiting embodiments or aspects, image capture system 102 may include an IR camera configured to capture and/or read identifier elements 114 including infrared and/or near-infrared fluorescent tags or markings. In some non-limiting embodiments or aspects, image capture system 102 may include a PTZ camera configured to use variable FOV and automatic zoom functions to automatically zoom in on and capture zoomed images of medical devices 112 and/or identifier elements 114 that are identified by image capture system 102 as objects for which a more detailed image is to be captured by the PTZ camera (e.g., identified as objects likely to be a medical device 112 and/or an identifier element 114, etc.). In some non-limiting embodiments or aspects, image capture system 102 may include a master and slave camera system including a static camera configured to capture an initial image(s) and a dynamic camera configured to zoom in on and capture zoomed images of medical devices 112 and/or identifier elements 114 that are identified by image capture system 102, based on the images from the static camera, as objects for which a more detailed image is to be captured (e.g., identified as objects likely to be a medical device 112 and/or an identifier element 114, etc.). In some non-limiting embodiments or aspects, image capture system 102 may include a color camera configured to capture and/or detect one or more predetermined wavelengths of light. In some non-limiting embodiments or aspects, image capture system 102 may include a camera including a filter configured to filter a predetermined wavelength of light to distinguish medical devices 112 and/or identifier elements 114 from a background or scene based on a color of the medical devices 112 and/or identifier elements 114 in the captured images.

Management system 104 may include one or more devices capable of receiving information and/or data from image capture system 102, user device 106, and/or auxiliary system 108 (e.g., via communication network 110, etc.) and/or communicating information and/or data to image capture system 102, user device 106, and/or auxiliary system 108 (e.g., via communication network 110, etc.). For example, management system 104 may include one or more computing systems including one or more processors (e.g., one or more computing devices, one or more server computers, one or more mobile computing devices, etc.). In some non-limiting embodiments or aspects, image capture system 102, management system 104, and/or user device 106 may be implemented within a single system or a single device.

Management system 104 (and/or image capture system 102, etc.) may be configured to obtain image data and process the image data to determine object data associated with objects detected and/or determined from the image data. For example, management system 104 may obtain image data from image capture system 102. As an example, management system 104 may obtain a plurality of images, captured over a period of time, of an environment (e.g., environment 100, etc.) surrounding one or more image capture devices of image capture system 102. In such an example, management system 104 may be configured to detect and/or determine, based on the images captured by image capture system 102 (e.g., based on image data, etc.), object data associated with at least one of the following: objects in the images (e.g., medical devices 112, identifier elements 114, medical devices 112 associated with identifier elements 114, etc.), types of the objects in the images, locations of the objects within environment 100 and/or with respect to other objects (e.g., other medical devices 112, other identifier elements 114, a patient, a caretaker, an image capture device, etc.), orientations (e.g., fluid flow path orientations through medical devices 112, inputs and outputs of medical devices 112, etc.) of the objects within environment 100 and/or with respect to the other objects, movements and/or trajectories of motion of the objects within environment 100 and/or with respect to the other objects, or any combination thereof. For example, management system 104 may determine, based on the detected and/or determined objects in the images, object data associated with at least one of the following: the types of the objects in the images, the locations of the objects within environment 100 and/or with respect to other objects, the orientations of the objects within environment 100 and/or with respect to the other objects, and/or the movements and/or trajectories of motion of the objects within environment 100 and/or with respect to the other objects, or any combination thereof.

In some non-limiting embodiments or aspects, management system 104 may differentiate medical devices 112 and/or identifier elements 114 from background and/or other objects in captured images based on object features detected and/or determined in the images, such as geometries of the medical devices 112 and/or the identifier elements 114, orientations in camera field-of-view of the medical devices 112 and/or the identifier elements 114, colors of the medical devices 112 and/or the identifier elements 114, proximities of the medical devices 112 and/or the identifier elements 114 to other medical devices 112 and/or other identifier elements 114, a patient, a caretaker, an image capture device, and/or the like, and/or interactive associations with the other medical devices 112 and/or the other identifier elements 114, the patient, the caretaker, the image capture device, and/or the like. For example, management system 104 may automatically document usage of medical devices 112 as the medical devices 112 and/or identifier elements 114 associated with the medical devices 112 are tracked within a field of view of the one or more image capture devices of image capture system 102 to provide event and/or usage-based guidance and alerts to caretakers, which may reduce complications during vascular access management assessment by continuously monitoring and updating usage information associated with the medical devices 112 and reducing errors associated with manual documentation.

In some non-limiting embodiments or aspects, management system 104 may process the image data using one or more object detection techniques (e.g., a deep learning technique, an image processing technique, an image segmentation technique, etc.) to identify or determine medical devices 112 and/or identifier elements 114 in the images of the image data and/or in the object data associated with the medical devices 112 and/or the identifier elements 114. For example, a deep learning technique may include a bounding box technique that generates a box label for objects (e.g., medical devices 112, identifier elements 114, etc.) of interest in images, an image masking technique (e.g., masked FRCNN (RCNN or CNN) that captures specific shapes of objects (e.g., medical devices 112, identifier elements 114, etc.) in images, a trained neural network that identifies objects (e.g., medical devices 112, identifier elements 114, etc.) in images, and/or the like. As an example, an image processing technique may include a cross correlation image processing technique, an image contrasting technique, a binary or colored filtering technique, and/or the like.

In some non-limiting embodiments or aspects, management system 104 may process the image data using a stereoscopic imaging technique and/or a shadow distance technique to determine object data including a distance from image capture system 102 to detected objects and/or distances between detected objects, and/or image capture system 102 may obtain the image data using multiple cameras, a laser focus technology, LiDAR sensors, and/or a camera physical zoom-in function to determine object data including a distance from image capture system 102 to detected objects and/or distances between detected objects. In some non-limiting embodiments or aspects, image capture system 102 may obtain image data and/or object data including a 3D profile of an object using a 3D optical profiler.

In some non-limiting embodiments or aspects, management system 104 may determine, based on image data and/or object data, event data associated with events and/or activities (e.g., vascular access management events, connections between medical devices 112, disconnections of medical devices 112, uses of medical devices, such as scrubbing events, disinfecting events, reuses of medical devices 112, replacements of medical devices 112, and/or the like, etc.) associated with the detected objects and/or an amount of time associated with the determined events and/or activities (e.g., an amount of time medical devices 112 are connected, a scrubbing time associated with a medical device 112, a drying time associated with a medical device after the scrubbing time, etc.).

In some non-limiting embodiments or aspects, management system 104 may generate, based on image data and/or object data (e.g., based on locations of detected medical devices 112 and/or identifier elements 114, types of the detected medical devices 112 and/or identifier elements 114, orientations of the detected medical devices 112 and/or identifier elements 114 relative to one another, movements and/or trajectories of the detected medical devices 112 and/or identifier elements 114 relative to one another, etc.), event data including a relational model of which medical devices 112 are connected to one another, as well as determine when these medical devices 112 are connected to each other and/or a duration of connection, disconnected from each other and/or a duration of disconnection, are involved in one or more other events or caretaker activities, and/or times associated therewith. For example, management system 104 may use one or more criteria, such as a threshold distance between points of medical devices 112, relative orientations and/or direction vectors of the medical devices 112, threshold times associated therewith, and/or the like, to determine event data associated with whether medical devices 112 are connected and/or disconnected from each other (e.g., whether a fluid path connection is established between medical devices 112, etc.), whether another event involving one or more of the medical devices 112 has occurred (e.g., a scrubbing or disinfecting event, etc.), and/or times associated therewith. In such an example, as medical devices 112 are moved in environment 100, continuing connections of the same medical devices 112 at or over different points in time (and/or in different images) may increase a probability that those medical devices are connected in a fluid pathway.

In some non-limiting embodiments or aspects, management system 104 may generate one or more models (e.g., an estimator, a classifier, a prediction model, a detector model, etc.) using one or more machine learning techniques including, for example, supervised and/or unsupervised techniques, such as decision trees (e.g., gradient boosted decision trees, random forests, etc.), logistic regressions, artificial neural networks (e.g., convolutional neural networks, etc.), Bayesian statistics, learning automata, Hidden Markov Modeling, linear classifiers, quadratic classifiers, association rule learning, and/or the like. Management system 104 may generate the model based on image data and/or object data (e.g., training data, etc.) associated with one or more environments. In some implementations, the model is designed to receive, as an input, image data and/or object data and provide, as an output, a prediction (e.g., a probability, a binary output, a yes-no output, a score, a prediction score, a classification, event data, etc.) as to whether one or more events have occurred. In some non-limiting embodiments, management system 104 stores the model (e.g., stores the model for later use). In some non-limiting embodiments or aspects, management system 104 may store the model in a data structure (e.g., a database, a linked list, a tree, etc.). In some non-limiting embodiments, the data structure is located within management system 104 or external (e.g., remote from) management system 104 (e.g., within autonomous vehicle 102, etc.). The one or more machine learning models may be trained to provide an output including event data associated with a prediction or classification of an event or activity associated with one or more medical devices 112 in response to input including image data and/or object data. In such an example, the prediction or classification of an event may include at least one of the following predictions or classifications: (i) a reuse of a medical device including a disconnection of the medical device from at least one of a patient and another medical device a reconnection of the medical device to the at least one of the patient and the another medical device, (ii) a replacement of a medical device with a new medical device of a same type as the medical device (e.g., a catheter dressing change, a needless connector change, etc.) (iii) a connection of a first medical device to a second medical device, (iv) a disconnection of a first medical device from a second medical device, (v) a scrubbing or disinfecting event including scrubbing or disinfecting a medical device with another medical device, (vi) a drying event including an amount of time a medical device remains disconnected from other medical devices after a scrubbing or disinfecting event, (vii) a medication delivery event including an amount and/or a type of a medication delivered to a patient and which lines, lumens, and/or medical devices are used to deliver the medication, (viii) a disposal event including disposal of a medication and/or a medical device in a disposal or waste device, or any combination thereof. In some non-limiting embodiments or aspects, a prediction or classification may include a probability score associated with a class prediction for an event. For example, the prediction or classification of the event may include a probability that the event occurred. As an example, the prediction or classification of the event may include at least one of the following: (i) a probability that a reuse of a medical device occurred (e.g., a reuse of a disinfectant swab or wipe, etc.), (ii) a probability that a replacement of a medical device occurred (e.g., a catheter dressing change, a needless connector change, etc.) (iii) a probability that a connection of a first medical device to a second medical device occurred, (iv) a probability that a disconnection of a first medical device from a second medical device occurred, (v) a probability that a scrubbing or disinfecting event occurred, (vi) a probability that a drying event occurred, or any combination thereof.

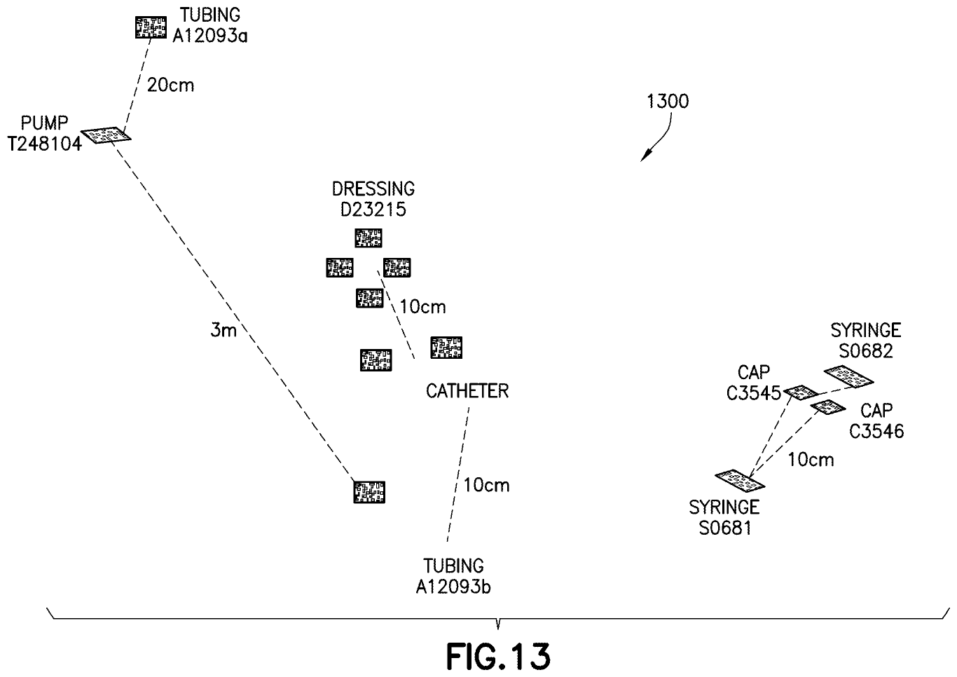

In some non-limiting embodiments or aspects, management system 104 may generate and/or update, based on the image data, the object data, and/or the event data, a database including locations of detected medical devices 112 and/or identifier elements 114, types of the detected medical devices 112 and/or identifier elements 114, orientations of the detected medical devices 112 and/or identifier elements 114 relative to one another, movements and/or trajectories of the detected medical devices 112 and/or identifier elements 114 relative to one another, which medical devices 112 are connected to one another, when medical devices 112 are connected to each other and/or a duration of connection, which medical devices 112 are disconnected from each other and/or a duration of disconnection, which medical devices 112 are involved in one or more other events or activities and/or a duration thereof, and/or times associated therewith. For example, management system 104 may maintain and update a database that includes spatial relationships of medical devices 112 and/or identifier elements 114 (e.g., distances between medical devices 112 and/or identifier elements 114, etc.) at a gross level (e.g., where an object is represented as a point, etc.) and orientations of the objects (e.g., fluid path vectors associated with a fluid path direction through a medical device, etc.), 3-space data that includes point locations of input-connectors and output connectors of medical devices 112, as well as intermediate points for larger devices, and/or the like, and/or determined events or activities therebetween. As an example, the database may include at least one of the following: a list of the plurality of medical devices currently in the environment, spatial relationships between the plurality of medical devices, current connections between the plurality of medical devices, current trajectories of the plurality of medical devices, orientations of the plurality of medical devices, events associated with the plurality of medical devices, or any combination thereof. In such an example, management system 104 may control a display (e.g., a display of user device 106, etc.) to display a visual representation of the information stored and/or maintained in the database. For example, FIG. 13 illustrates an example visual representation 1300 of the implementation of environment 100 shown in FIG. 1B. As shown in FIG. 13, management system 104 may represent detected medical devices 112 and/or identifier elements 114 may be as points associated with identifiers and spatial distances between the medical devices 112 and/or identifier elements 114.

User device 106 may include one or more devices capable of receiving information and/or data from image capture system 102, management system 104, and/or auxiliary system 108 (e.g., via communication network 110, etc.) and/or communicating information and/or data to image capture system 102, management system 104, and/or auxiliary system 108 (e.g., via communication network 110, etc.). For example, user device 106 may include one or more computing systems including one or more processors (e.g., one or more computing devices, one or more server computers, one or more mobile computing devices, etc.). In some non-limiting embodiments or aspects, user device 106 may be configured to obtain and/or display image data, object data, and/or event data from management system 104. For example, as shown in FIG. 13, user device 106 may be configured to display spatial relationships of medical devices 112 and/or identifier elements 114 with the medical devices 112 and/or identifier elements 114 represented as points in the display indicating the locations or most probable locations of the medical devices 112 and/or identifier elements 114 determined by management system 104, indications of connections between the medical devices 112 and/or identifier elements 114 represented as points, and/or indication of orientations of the medical devices 112 and/or identifier elements 114 represented as points. As an example, user device 106 may be configured to automatically provide, in response to a determination of an event associated with one or more medical devices, an alert associated with the determined event (e.g., display instructions or a warning associated with event, emit audible instructions or warnings associated with the event, etc.).

In some non-limiting embodiments or aspects, image capture system 102 may be implemented within user device 106. For example, user device 106 may include a tablet computer including one or more cameras.

In some non-limiting embodiments or aspects, user device 106 includes a nurse station or terminal in a hospital. For example, user device 106 may provide bedside nurse support (e.g., recordation of events in real-time by a nurse and feedback to a nurse if events, such as scrubbing or flushing, are determined to be due or needed, etc.), nursing station manager support (e.g., optimization of flushing procedures to reduce workflow and improve timed targets for flushing, etc.), retrospective reporting for nursing administration (e.g., a scrub duration, a flushing technique, a time between flushes, improper medical device reuse, proper medical device replacement, etc.), and/or the like.

Auxiliary device 108 may include one or more devices capable of receiving information and/or data from image capture system 102, management system 104, and/or user device 106 (e.g., via communication network 110, etc.) and/or communicating information and/or data to image capture system 102, management system 104, and/or user device 106 (e.g., via communication network 110, etc.). For example, auxiliary device 108 may include one or more computing systems including one or more processors (e.g., one or more computing devices, one or more server computers, one or more mobile computing devices, etc.).

In some non-limiting embodiments or aspects, auxiliary device 108 may include one or more image capture devices and/or one or more audio capture devices (e.g., microphones, etc.), which may be separate and distinct from image capture system 102. For example, auxiliary device 108 may include one or more image capture devices configured to capture one or more images of environment 100 and/or one or more audio capture devices configured to capture audio within environment 100 and/or provide the captured images and/or audio to management system 104 as auxiliary data. Accordingly, management system 104 may augment image data captured by image capture system 102 with auxiliary data from auxiliary device 108 to more accurately resolve situations with higher environmental noise and/or occlusions to a field of view of image capture system 102, which may enable better documentation of vascular access management activities and/or better patient treatment. For example, management system 104 may use auxiliary data including images to determine a size, a type, a shape, and/or a location of a medical device (e.g., a catheter or tubing, etc.) which may be obscured (e.g., by a catheter dressing, etc.) in images captured by image capture device 102. As an example, management system 104 may use audio location technology (e.g., Sonar, etc.) to process auxiliary data including audio to determine locations of devices associated with predetermined audio signals captured in the auxiliary data. In such an example, an infusion pump may emit a distinct noise that can be captured and used to identify the infusion pump and/or determine a location of the infusion pump in environment 100.

In some non-limiting embodiments or aspects, auxiliary device 108 may include a secure hospital server and/or one or more secure hospital databases that store electronic health records (EHRs), personally identifiable information (PII), and/or Health Insurance Portability and Accountability Act (HIPAA) protected information. For example, auxiliary device 108 may provide auxiliary data including information from an EHR (e.g., a type of medical device prescribed to a patient in environment 100, a location prescribed for use of the medical device, such as a catheter insertion site on the patent, etc.) to management system 104, and/or management system 104 may use the auxiliary data including the information from the EHR, etc. to determine a size, a type, a shape, and/or a location of a medical device 112 associated with the patient.

Communication network 110 may include one or more wired and/or wireless networks. For example, communication network 110 may include a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

The number and arrangement of systems and devices shown in FIGS. 1A and 1B are provided as an example. There can be additional systems and/or devices, fewer systems and/or devices, different systems and/or devices, or differently arranged systems and/or devices than those shown in FIGS. 1A and 1B. Furthermore, two or more systems or devices shown in FIGS. 1A and 1B can be implemented within a single system or a single device, or a single system or a single device shown in FIGS. 1A and 1B can be implemented as multiple, distributed systems or devices. Additionally, or alternatively, a set of systems or a set of devices (e.g., one or more systems, one or more devices, etc.) of environment 100 can perform one or more functions described as being performed by another set of systems or another set of devices of environment 100.

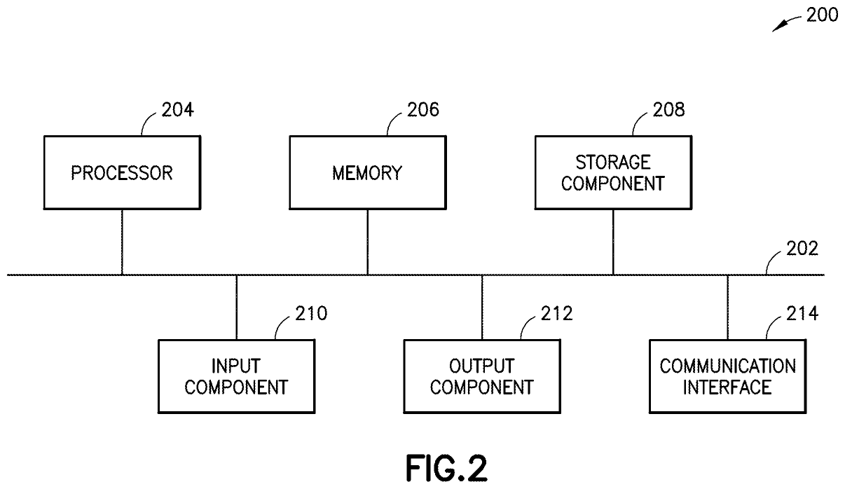

Referring now to FIG. 2, FIG. 2 is a diagram of example components of a device 200. Device 200 may correspond to one or more devices of image capture system 102, one or more devices of management system 104, user device 106 (e.g., one or more devices of a system of user device 106, etc.), and/or one or more devices of auxiliary system 108. In some non-limiting embodiments or aspects, one or more devices of image capture system 102, one or more devices of management system 104, user device 106 (e.g., one or more devices of a system of user device 106, etc.), and/or one or more devices of auxiliary system 108 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

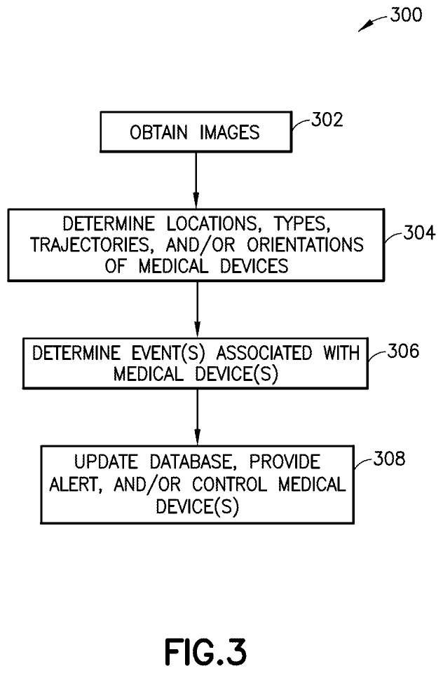

Referring now to FIG. 3, FIG. 3 is a flowchart of non-limiting embodiments or aspects of a process 300 for vascular access management. In some non-limiting embodiments or aspects, one or more of the steps of process 300 may be performed (e.g., completely, partially, etc.) by management system 104 (e.g., one or more devices of management system 104, etc.). In some non-limiting embodiments or aspects, one or more of the steps of process 300 may be performed (e.g., completely, partially, etc.) by another device or a group of devices separate from or including management system 104, such as image capture system 102 (e.g., one or more devices of image capture system, etc.), user device 106 (e.g., one or more devices of a system of user device 106, etc.), and/or auxiliary system 108 (e.g., one or more devices of auxiliary system 108, etc.).

As shown in FIG. 3, at step 302, process 300 includes obtaining images. For example, management system 104 may obtain images. As an example, one or more image capture devices of image capture system 102 may capture, over a period of time, a plurality of images of an environment (e.g., environment 100, etc.) surrounding the one or more image capture devices. In such an example, management system 104 may obtain the plurality of images from image capture system 102.

As shown in FIG. 3, at step 304, process 300 includes determining locations, types, trajectories, and/or orientations of medical devices. For example, management system 104 may determine, based on the plurality of images, a plurality of locations of a plurality of medical devices 112 within the environment over the period of time, a plurality of types of the plurality of medical devices 112, a plurality of trajectories of the plurality of medical devices 112 within the environment over the period of time, and/or a plurality of orientations of the plurality of medical devices 112 within the environment over the period of time.

As shown in FIG. 3, at step 306, process 300 includes determining at least one event associated with at least one medical device. For example, management system 104 may determine, based on the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time, at least one event associated with at least one medical device of the plurality of medical devices 112.

In some non-limiting embodiments or aspects, the at least one event may include a connection between two or more medical devices of the plurality of medical devices 112. For example, management system 104 may determine, based on the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time, at least one connection between two or more medical devices of the plurality of medical devices 112.

In some non-limiting embodiments or aspects, at least one connection between two or more medical devices forms a fluid flow path through the two or more medical devices. For example, management system 104 may determine, based on the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time, at least one event including at least one connection between two or more medical devices of the plurality of medical devices 112 that forms a fluid flow path through the two or more medical devices and a direction of a fluid flow in the fluid plow path through the two or more medical devices.

In some non-limiting embodiments or aspects, the at least one event may include at least one event of the following events: (i) a reuse of a medical device including a disconnection of the medical device from at least one of a patient and another medical device in the environment and a reconnection of the medical device to the at least one of the patient and the another medical device in the environment (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device. For example, management device 104 may determine, based on the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time, at least one event of the following events: (i) a reuse of a medical device including a disconnection of the medical device from at least one of a patient and another medical device in the environment and a reconnection of the medical device to the at least one of the patient and the another medical device in the environment, (ii) a replacement of the medical device with a new medical device of a same type as the medical device in the environment including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device.

As shown in FIG. 3, at step 308, process 300 includes updating a database, providing an alert, and/or controlling one or more medical devices. For example, management system 104 may update a database, provide an alert, and/or control at least one medical device based on the at least one event associated with the at least one medical device, the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time.

In some non-limiting embodiments or aspects, in response to the at least one event including a reuse of a medical device, management system 104 may at least one of: provide to user device 106, an alert associated with the use of the medical device 112; and automatically control at least one medical device (e.g., a valve, an infusion pump, etc.) to stop a flow of fluid in a fluid flow path including the medical device.

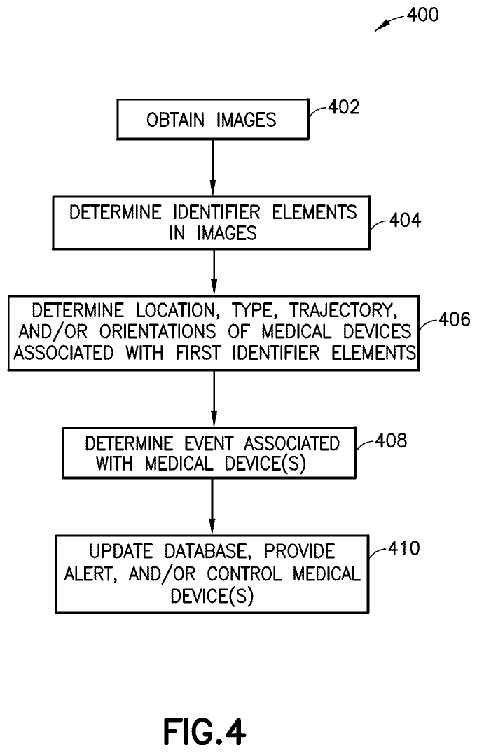

Referring now to FIG. 4, FIG. 4 is a flowchart of non-limiting embodiments or aspects of a process 400 for vascular access management. In some non-limiting embodiments or aspects, one or more of the steps of process 400 may be performed (e.g., completely, partially, etc.) by management system 104 (e.g., one or more devices of management system 104, etc.). In some non-limiting embodiments or aspects, one or more of the steps of process 300 may be performed (e.g., completely, partially, etc.) by another device or a group of devices separate from or including management system 104, such as image capture system 102 (e.g., one or more devices of image capture system, etc.), user device 106 (e.g., one or more devices of a system of user device 106, etc.), and/or auxiliary system 108 (e.g., one or more devices of auxiliary system 108, etc.).

As shown in FIG. 4, at step 402, process 300 includes obtaining images. For example, management system 104 may obtain images. As an example, one or more image capture devices of image capture system 102 may capture, over a period of time, a plurality of images of an environment (e.g., environment 100, etc.) surrounding the one or more image capture devices. In such an example, management system 104 may obtain the plurality of images from image capture system 102.

As shown in FIG. 4, at step 404, process 300 includes determining identifier elements in images. For example, management system 104 may determine, based on the plurality of images, a plurality of identifier elements 114 within the environment over the period of time. As an example, the plurality of identifier elements 114 may be associated with a plurality of medical devices 112, and the plurality of identifier elements 114 may encapsulate a plurality of identifiers associated a plurality of types of the plurality of medical devices 112. In some non-limiting embodiments or aspects, the plurality of identifiers may uniquely identify the plurality of medical devices 112 from each other.

In some non-limiting embodiments or aspects, the plurality of identifier elements 114 may include at least one identifier element including a fluorescent coating configured to emit light of a predetermined wavelength, and image capture system 102 may capture only the light of the predetermined wavelength in the plurality of images.

In some non-limiting embodiments or aspects, the plurality of identifier elements 114 includes at least one identifier element including at least one LED configured to emit light of at least one predetermined wavelength in at least one pattern and/or at least one intensity, and image capture system 102 may capture only the light of the predetermined wavelength in the plurality of images. For example, management system 104 may determine, based on the at least one pattern and/or the at least one intensity of the emitted light of the at least one predetermined wavelength captured in the plurality of images, a type of a medical device associated with the at least one identifier element.

In some non-limiting embodiments or aspects, the plurality of identifier elements 114 includes at least one identifier element including at least one color changing dye configured to change color over the period of time. For example, management system 104 may determine, based on a change in the color of the color changing dye in the plurality of images, an amount of time associated with a use of a medical device associated with the at least one identifier element.

As shown in FIG. 4, at step 406, process 300 includes determining locations, types, trajectories, and/or orientations of medical devices. For example, management system 104 may determine, based on the plurality of identifier elements determined in the plurality of images, a plurality of locations of the plurality of medical devices 112 within the environment over the period of time, a plurality of types of the plurality of medical devices 112, a plurality of trajectories of the plurality of medical devices 112 within the environment over the period of time, and/or a plurality of orientations of the plurality of medical devices 112 within the environment over the period of time.

As shown in FIG. 4, at step 408, process 300 includes determining at least one event associated with at least one medical device. For example, management system 104 may determine, based on the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time, at least one event associated with at least one medical device of the plurality of medical devices 112.

As shown in FIG. 4, at step 410, process 300 includes updating a database, providing an alert, and/or controlling one or more medical devices. For example, management system 104 may update a database, provide an alert, and/or control at least one medical device based on the at least one event associated with the at least one medical device, the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time.

In some non-limiting embodiments or aspects, in response to the at least one event including a reuse of a medical device, management system 104 may at least one of: provide to user device 106, an alert associated with the use of the medical device 112; and automatically control at least one medical device (e.g., a valve, an infusion pump, etc.) to stop a flow of fluid in a fluid flow path including the medical device.

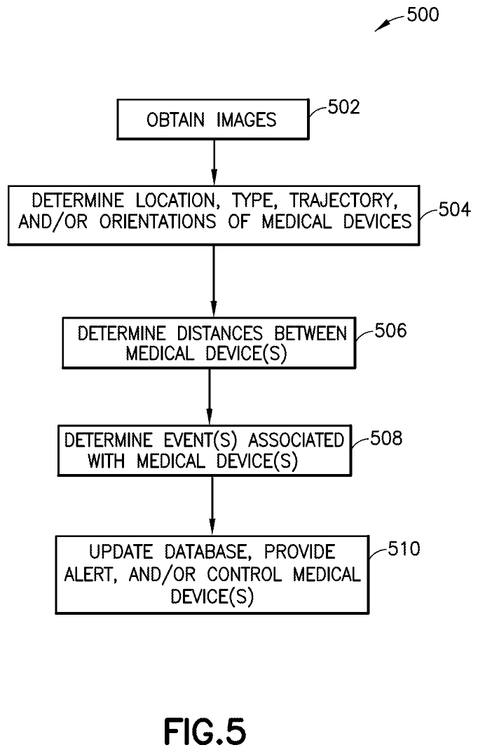

Referring now to FIG. 5, FIG. 5 is a flowchart of non-limiting embodiments or aspects of a process 500 for vascular access management. In some non-limiting embodiments or aspects, one or more of the steps of process 500 may be performed (e.g., completely, partially, etc.) by management system 104 (e.g., one or more devices of management system 104, etc.). In some non-limiting embodiments or aspects, one or more of the steps of process 500 may be performed (e.g., completely, partially, etc.) by another device or a group of devices separate from or including management system 104, such as image capture system 102 (e.g., one or more devices of image capture system, etc.), user device 106 (e.g., one or more devices of a system of user device 106, etc.), and/or auxiliary system 108 (e.g., one or more devices of auxiliary system 108, etc.).

As shown in FIG. 5, at step 502, process 500 includes obtaining images. For example, management system 104 may obtain images. As an example, image capture system 102 may capture, over a period of time, a plurality of images of an environment (e.g., environment 100, etc.) surrounding the one or more image capture devices. In such an example, management system 104 may obtain the plurality of images from image capture system 102.

As shown in FIG. 5, at step 504, process 500 includes determining locations, types, trajectories, and/or orientations of medical devices. For example, management system 104 may determine, based on the plurality of images, a plurality of locations of a plurality of medical devices 112 within the environment over the period of time, a plurality of types of the plurality of medical devices 112, a plurality of trajectories of the plurality of medical devices 112 within the environment over the period of time, and/or a plurality of orientations of the plurality of medical devices 112 within the environment over the period of time.

As shown in FIG. 5, at step 506, process 500 includes determining distances between medical devices. For example, management system 104 may determine, based on the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time, a plurality of distances between the plurality of medical devices 112 over the period of time.

In some non-limiting embodiments or aspects, management system 104 may delay determining the plurality of distances between the plurality of medical devices 112 over the period of time and determining the at least one event until a location of at least one of the first medical device and the second medical device changes in the plurality of images over the period of time. Accordingly, management system 104 may save processing power and/or other computer resources until they are needed to detect a change in environment 100.

As shown in FIG. 5, at step 508, process 500 includes determining at least one event associated with at least one medical device. For example, management system 104 may determine, based on the plurality of distances between the plurality of medical devices 112 over the period of time and the plurality of types of the plurality of medical devices 112 (and/or the plurality of locations of the plurality of medical devices 112 within the environment over the period of time, the plurality of trajectories of the plurality of medical devices 112 within the environment over the period of time, and/or the plurality of orientations of the plurality of medical devices 112 within the environment over the period of time), at least one event associated with at least one medical device of the plurality of medical devices 112. As an example, management system 104 may determine, based on the plurality of distances between the plurality of medical devices 112 over the period of time and the plurality of types of the plurality of medical devices 112, at least one event of the following events: (i) a connection of a first medical device of the plurality of medical devices 112 to a second medical device of the plurality of medical devices 112 and (ii) a disconnection of the first medical device of the plurality of medical devices 112 from the second medical device of the plurality of medical devices 112. In some non-limiting embodiments or aspects, determining the at least one event further determines a probability associated with the at least one event.

In some non-limiting embodiments or aspects, management system 104 may determine based on the plurality of distances between the plurality of medical devices 112 over the period of time and the plurality of types of the plurality of medical devices 112, one of the following further events: (i) a reuse of the first medical device including a disconnection of the first medical device from the second medical device in the environment and a reconnection of the first medical device to the second medical device in the environment, (ii) a replacement of the first medical device with a new medical device of a same type as the first medical device including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device.

In some non-limiting embodiments or aspects, management system 104 may determine, based on an orientation of the first medical device and an orientation of the second medical device, a direction of a fluid flow in a fluid plow path through the first medical device and the second medical device and/or update, based on the direction of the fluid flow in the fluid plow path through the first medical device and the second medical device, a database.

In some non-limiting embodiments or aspects, the first medical device includes at least one of a disinfectant cap and a disinfectant swab such that the connection of the first medical device of the plurality of medical devices 112 to the second medical device of the plurality of medical devices 112 does not form a fluid flow path through the first medical device and the second medical device. For example, the connection of the first medical device of the plurality of medical devices 112 to the second medical device of the plurality of medical devices 112 may be associated with a scrubbing event including scrubbing of the second medical device (e.g., a needless connector, etc.) with the disinfectant cap and/or the disinfectant swab.

As shown in FIG. 5, at step 510, process 500 includes updating a database, providing an alert, and/or controlling one or more medical devices. For example, management system 104 may update a database, provide an alert, and/or control at least one medical device based on the at least one event associated with the at least one medical device, the plurality of locations of the plurality of medical devices 112 within the environment over the period of time, the plurality of types of the plurality of medical devices 112, the plurality of trajectories of the plurality of medical devices 112 within the environment over the period of time, and/or the plurality of orientations of the plurality of medical devices 112 within the environment over the period of time.

In some non-limiting embodiments or aspects, in response to the at least one event including a reuse of a medical device, management system 104 may at least one of: provide to user device 106, an alert associated with the use of the medical device 112; and automatically control at least one medical device (e.g., a valve, an infusion pump, etc.) to stop a flow of fluid in a fluid flow path including the medical device.

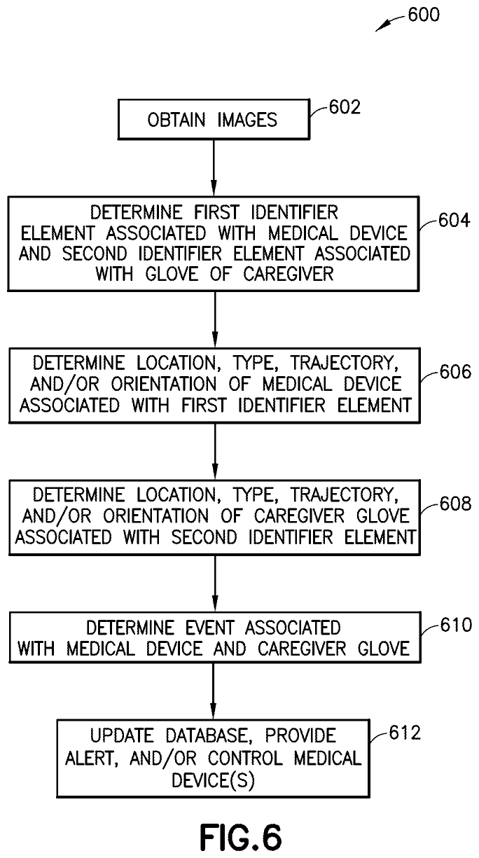

Referring now to FIG. 6, FIG. 6 is a flowchart of non-limiting embodiments or aspects of a process 600 for vascular access management. In some non-limiting embodiments or aspects, one or more of the steps of process 600 may be performed (e.g., completely, partially, etc.) by management system 104 (e.g., one or more devices of management system 104, etc.). In some non-limiting embodiments or aspects, one or more of the steps of process 500 may be performed (e.g., completely, partially, etc.) by another device or a group of devices separate from or including management system 104, such as image capture system 102 (e.g., one or more devices of image capture system, etc.), user device 106 (e.g., one or more devices of a system of user device 106, etc.), and/or auxiliary system 108 (e.g., one or more devices of auxiliary system 108, etc.).

As shown in FIG. 6, at step 602, process 600 includes obtaining images. For example, management system 104 may obtain images. As an example, image capture system 102 may capture, over a period of time, a plurality of images of an environment (e.g., environment 100, etc.) surrounding the one or more image capture devices. In such an example, management system 104 may obtain the plurality of images from image capture system 102.

As shown in FIG. 6, at step 604, process 600 includes determining a first identifier element associated with a medical device and a second identifier element associated with a glove of a caregiver. For example, management system 104 may determine, based on the plurality of images, a first identifier element associated with a medical device (e.g., a needless connector, etc.) and a second identifier element associated with a glove of a caregiver. As an example, the first identifier element may encapsulate a first identifier associated with the medical device, and the second identifier element may encapsulate a second identifier associated with the glove of the caregiver.

As shown in FIG. 6, at step 606, process 600 includes determining locations, types, trajectories, and/or orientations of the medical device associated with the first identifier element. For example, management system 104 may determine, based on the first identifier element in the plurality of images, the medical device and a location, a type, a trajectory, and/or an orientation of the medical device within the environment over the period of time.

As shown in FIG. 6, at step 608, process 600 includes determining locations, types, trajectories, and/or orientations of the caregiver glove associated with the second identifier element. For example, management system 104 may determine, based on the second identifier element in the plurality of images, the glove of the caregiver and a location, a type, a trajectory, and/or an orientation of the glove of the caregiver within the environment over the period of time. As an example, the second identifier may include a predetermined color of the glove of the caregiver.

As shown in FIG. 6, at step 610, process 600 includes determining at least one event associated with the medical device and the caregiver glove. For example, management system 104 may determine, based on the location, the type, the trajectory, and/or the orientation of the medical device within the environment over the period of time and the location, the type, the trajectory, and/or the orientation of the glove of the caregiver within the environment over the period of time and the location, at least one event associated with the medical device. In such an example, the at least one event may include a catheter dressing change event including replacing the medical device (e.g., a catheter dressing, etc.) with a new medical device of the same type.

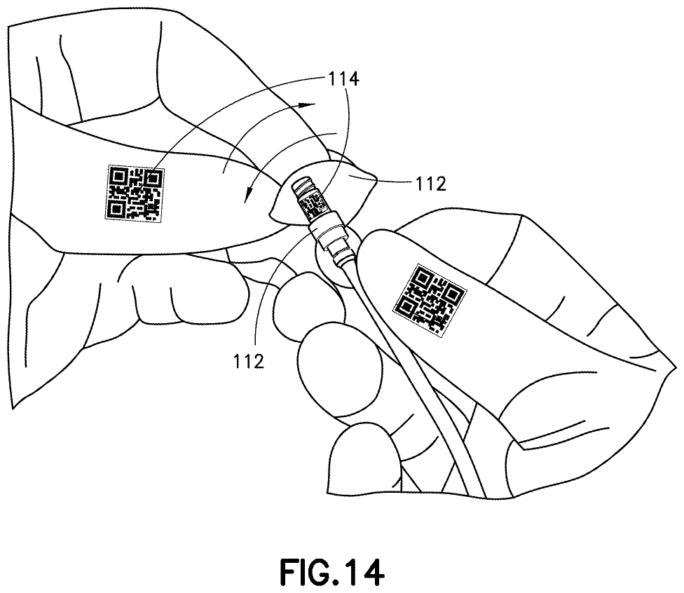

In some non-limiting embodiments or aspects, and referring also to FIG. 14, management system 104 may determine, based on the plurality of images, a location, a type, a trajectory, and/or an orientation of a further medical device (e.g., a disinfectant wipe, etc.) within the environment over the period of time. For example, management system 104 may determine, based on the location, the type, the trajectory, and/or the orientation of the further medical device over the period of time and at least one of (i) the location, the type, the trajectory, and/or the orientation of medical device within the environment over the period of time and (ii) the location, the type, the trajectory, and/or the orientation of the glove of the caregiver within the environment over the period of time, a linear distance change and an angular distance change between the further medical device and the at least one of the medical device and the glove of the caregiver over the period of time. As an example, management system 104 may further determine the at least one event and/or a duration of the at least one event based on the linear distance change and the angular distance change between the medical device and the glove of the caregiver. In such an example, as shown in FIG. 14, the at least one event may include a scrubbing or disinfecting event including scrubbing of the medical device (e.g., a needless connector etc.) held in the glove of the caregiver with the further medical device (e.g., a disinfectant swab or wipe, etc.) held in the other glove of the caregiver.

As shown in FIG. 6, at step 612, process 600 includes updating a database, providing an alert, and/or controlling one or more medical devices. For example, management system 104, based on the at least one determined event, update a database including events associated with the environment, provide an alert associated with the at least one determined event, and/or control one or more medical devices in the environment.

In some non-limiting embodiments or aspects, in response to the at least one event, management system 104 may at least one of: provide to user device 106, an alert associated with the at least one event; and automatically control at least one medical device (e.g., a valve, an infusion pump, etc.) to stop a flow of fluid in a fluid flow path including the medical device associated with the at least one event.

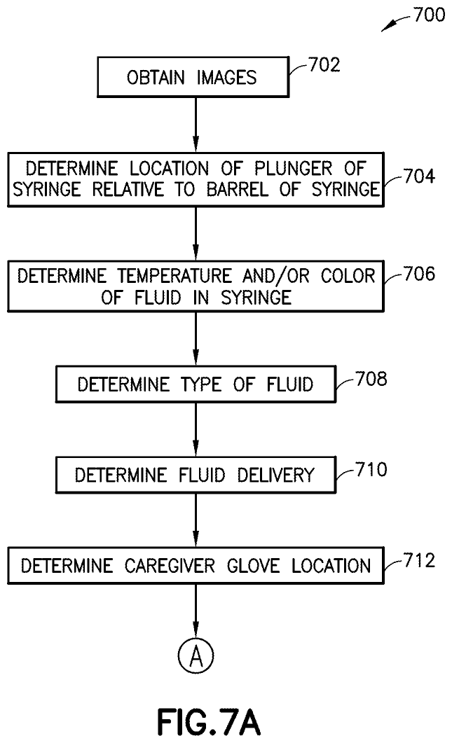

Referring now to FIGS. 7A and 7B, FIGS. 7A and 7B are a flowchart of non-limiting embodiments or aspects of a process 700 for vascular access management. In some non-limiting embodiments or aspects, one or more of the steps of process 700 may be performed (e.g., completely, partially, etc.) by management system 104 (e.g., one or more devices of management system 104, etc.). In some non-limiting embodiments or aspects, one or more of the steps of process 700 may be performed (e.g., completely, partially, etc.) by another device or a group of devices separate from or including management system 104, such as image capture system 102 (e.g., one or more devices of image capture system, etc.), user device 106 (e.g., one or more devices of a system of user device 106, etc.), and/or auxiliary system 108 (e.g., one or more devices of auxiliary system 108, etc.).

As shown in FIG. 7A, at step 702, process 700 includes obtaining images. For example, management system 104 may obtain images. As an example, image capture system 102 may capture, over a period of time, a plurality of images of an environment (e.g., environment 100, etc.) surrounding the one or more image capture devices. In such an example, management system 104 may obtain the plurality of images from image capture system 102.

As shown in FIG. 7A, at step 704, process 700 includes determining a location of a plunger of a syringe relative to a barrel of the syringe. For example, management system 104 may determine, based on the plurality of images, a location of a plunger of a syringe relative to a barrel of the syringe in the environment over the period of time.

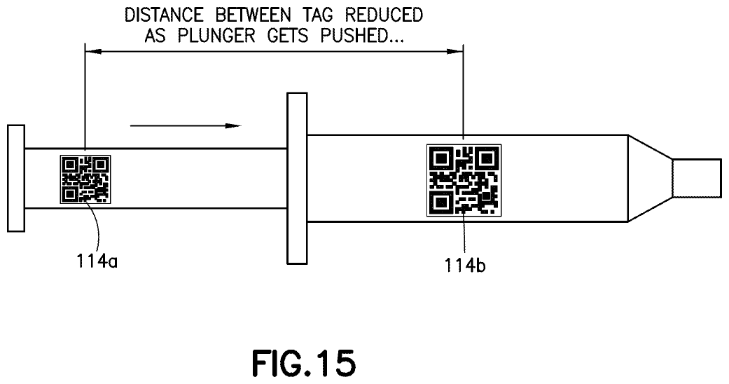

In some non-limiting embodiments or aspects, and referring also to FIG. 15, management system 104 may determine the location of the plunger of the syringe relative to the barrel of the syringe over the period of time further by determining, based on the plurality of images, a first identifier element 114a associated with the plunger of the syringe and a second identifier element 114b associated with the barrel of the syringe, and the location of the plunger of a syringe relative to a barrel of the syringe in the environment over the period of time is determined based on the first identifier element 114a in the plurality of images and the second identifier element 114b in the plurality of images. For example, determining the location of the plunger of the syringe relative to the barrel of the syringe in the environment over the period of time may include determining, based on the first identifier element 114a and the second identifier element 114b in the plurality of images, a distance between the first identifier element 114a and the second identifier element 114b as a distance between the plunger and the barrel of the syringe.

As shown in FIG. 7A, at step 706, process 700 includes determining a temperature and/or a color of a fluid in the syringe. For example, management system 104 may determine, based on the plurality of images, a temperature and/or a color of a fluid contained in the syringe. As an example, image capture system 102 may include a camera with a filter configured to capture the color in images and/or an IR camera configured to capture IR images.

As shown in FIG. 7A, at step 708, process 700 includes determining a type of the fluid. For example, management system 104 may determine, based on the fluid flow rate of a fluid delivery and/or the temperature and/or the color of the fluid, a type of the fluid associated with at least one fluid delivery. In some non-limiting embodiments or aspects, management system 104 may compare the temperature of the fluid contained in the syringe to a threshold temperature associated with the fluid (e.g., associated with the type of fluid, etc.) and, in response to determining that the temperature of the fluid contained in the syringe satisfies the threshold temperature, automatically control at least one medical device (e.g., an electronic valve, an infusion pump, etc.) to stop at least one fluid delivery from the syringe.

As shown in FIG. 7A, at step 710, process 700 includes determining a fluid delivery. For example, management system 104 may determine, based on the location of the plunger of the syringe relative to the barrel of the syringe over the period of time, at least one fluid delivery from the syringe. In some non-limiting embodiments or aspects, determining the least one fluid delivery further includes determining, based on the location of the plunger of the syringe relative to the barrel of the syringe over the period of time, at least one of an amount of fluid delivered by the at least one fluid delivery and a fluid flow rate of the at least one fluid delivery.

As shown in FIG. 7A, at step 712, process 700 includes determining a location of a glove of a caregiver. For example, management system 104 may determine, based on the plurality of images, a location of a glove of a caregiver within the environment over the period of time.

As show in FIG. 7B, at step 714, process 700 includes determining a flush technique associated with the fluid delivery. For example, management system 104 may determine, based on the location of the glove of the caregiver within the environment over the period of time, a flush technique associated with the at least one fluid delivery, wherein the flush technique includes a pulsatile flush or a continuous flush.

As shown in FIG. 7B, at step 716, process 700 includes updating a database, providing an alert, and/or controlling one or more medical devices. For example, management system 104, based on the at least one determined event (e.g., a fluid delivery, etc.), update a database including events associated with the environment, provide an alert associated with the at least one determined event, and/or control one or more medical devices in the environment.

In some non-limiting embodiments or aspects, in response to the at fluid delivery, management system 104 may at least one of: provide, to user device 106, an alert associated with the fluid delivery; and automatically control at least one medical device (e.g., a valve, an infusion pump, etc.) to stop a flow of fluid in a fluid flow path including the medical device associated with the at least one event.

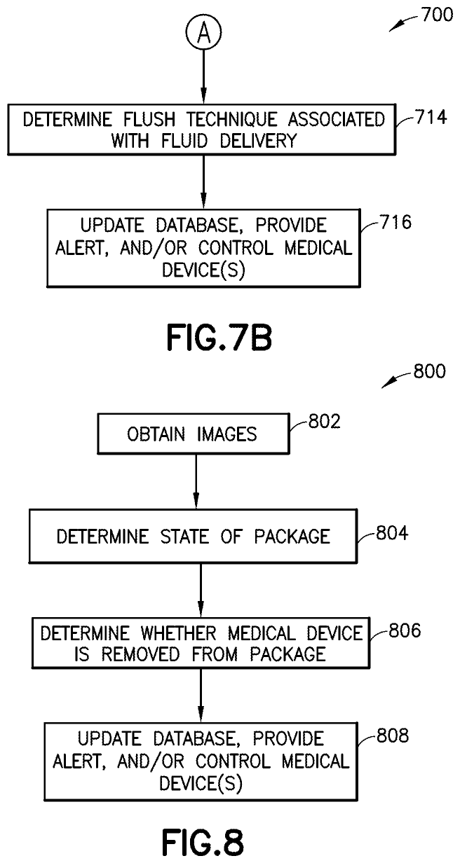

Referring now to FIG. 8, FIG. 8 is a flowchart of non-limiting embodiments or aspects of a process 800 for vascular access management. In some non-limiting embodiments or aspects, one or more of the steps of process 800 may be performed (e.g., completely, partially, etc.) by management system 104 (e.g., one or more devices of management system 104, etc.). In some non-limiting embodiments or aspects, one or more of the steps of process 800 may be performed (e.g., completely, partially, etc.) by another device or a group of devices separate from or including management system 104, such as image capture system 102 (e.g., one or more devices of image capture system, etc.), user device 106 (e.g., one or more devices of a system of user device 106, etc.), and/or auxiliary system 108 (e.g., one or more devices of auxiliary system 108, etc.).

As shown in FIG. 8, at step 802, process 800 includes obtaining images. For example, management system 104 may obtain images. As an example, image capture system 102 may capture, over a period of time, a plurality of images of an environment (e.g., environment 100, etc.) surrounding the one or more image capture devices. In such an example, management system 104 may obtain the plurality of images from image capture system 102.

As shown in FIG. 8, at step 804, process 800 includes determining a state of a package. For example, management system 104 may determine, based on the plurality of images, a state of a package containing a medical device over the period of time. As an example, a state of a package may include

As shown in FIG. 8, at step 806, process 800 includes determining whether a medical device is removed from the package. For example, management system 104 may determine, based on the state of the package over the period of time, whether the medical device is removed from the package. As an example, management system 104 may determine, based on the state of the package, a type of medical device 112 included in the package and whether the medical device 112 has been removed from the package. In such an example, management system 104 may determine, based on the type of the medical device and a time at which the medical device 112 is determined to be removed from the package, at least one event associated with the medical device, such as a first use of the medical device, a reuse of the medical device, a replacement of the medical device with a new medical device and/or a different type of medical device, and/or the like.

In some non-limiting embodiments or aspects, the package includes a removable first layer covering a second layer, wherein the first layer including a first color, wherein the second layer includes a second color different than the first color, and wherein removal of the first layer from the package reveals the second layer. In some non-limiting embodiments or aspects, a color of the package is configured to change when exposed to air. For example, management system 104 may determine, based on a detected or determined color or layer of the package, whether the package has been opened and the medical device removed from the package. In such an example, the first layer may be at least partially transparent.

In some non-limiting embodiments or aspects, a portion of the package is transparent such that the medical device contained within the package is visible through the transparent portion of the package. For example, management system 104 may determine, based on whether the medical device is detected or determined within the package, whether the package has been opened and the medical device removed from the package.

In some non-limiting embodiments or aspects, the package is associated with a first identifier element, the medical device is associated with a second identifier element different than the first identifier element, and the state of the package is determined based on a location of the first identifier element with respect to a location of the second identifier element. For example, management system 104 may determine, based on a distance between the first identifier element and the second identifier element satisfying a threshold distance, whether the package has been opened and the medical device removed from the package.

In some non-limiting embodiments or aspects, management device 104 may determine, based on the plurality of images, a location of a glove of a caregiver within the environment over the period of time with respect to a location of the package, and determine the state of the package based on the location of the glove of the caregiver within the environment over the period of time with respect to the location of the package.

In some non-limiting embodiments or aspects, the package includes a removable first layer covering a second layer, the removable first layer includes a first identifier element, the second layer includes a second identifier element, and the removable first layer is at least partially transparent. For example, management system 104 may determine the state of the package by: determining, based on the plurality of images, a distance between the first identifier element and the second identifier element; determining, based on the distance between the first identifier element and the second identifier element, whether the package is faulty; and in response to determining that the package is faulty, provide, to a user device, an alert associated with the faulty package.

In some non-limiting embodiments or aspects, management system 104 may determine, based on the plurality of images, a plurality of locations of a plurality of medical devices 112 within the environment over the period of time and a plurality of types of the plurality of medical devices 112; and determine, based on the plurality of locations of the plurality of medical devices 112 within the environment over the period of time, the plurality of types of the plurality of medical devices 112, and the state of the package over the period of time, at least one event of the following events: (i) a reuse of the medical device including a connection of the medical device to two or more medical devices in the environment over the period of time, (ii) a replacement of the medical device with a new medical device of a same type as the medical device in the environment, and (iii) a replacement of the medical device with different medical device of a different type than the medical device in the environment.

As shown in FIG. 8, at step 808, process 800 includes updating a database, providing an alert, and/or controlling one or more medical devices. For example, management system 104 may update, based on a determination that the medical device is removed from the package, a database including medical devices in the environment. As an example, management system 104 may determine, based on a determination that the medical device is removed from the package and/or a time associated therewith, whether an event associated with the medical device includes a first use of the medical device or a reuse of the medical device. In such an example, management system 104 may automatically document the first use and any reuse of a medical device, alert a nurse (e.g., via user device 106, etc.) that the medical device is being improperly reused, and/or control a medical device (e.g., a valve, an infusion pump, etc.) to stop of flow of fluid through a fluid flow path associated with the reused medical device, which may improve patient safety and/or reduce costs associated with complications arising from reuse of medical devices.

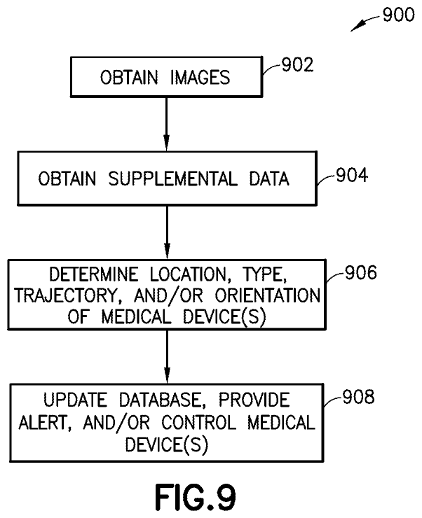

Referring now to FIG. 9, FIG. 9 is a flowchart of non-limiting embodiments or aspects of a process 900 for vascular access management. In some non-limiting embodiments or aspects, one or more of the steps of process 900 may be performed (e.g., completely, partially, etc.) by management system 104 (e.g., one or more devices of management system 104, etc.). In some non-limiting embodiments or aspects, one or more of the steps of process 900 may be performed (e.g., completely, partially, etc.) by another device or a group of devices separate from or including management system 104, such as image capture system 102 (e.g., one or more devices of image capture system, etc.), user device 106 (e.g., one or more devices of a system of user device 106, etc.), and/or auxiliary system 108 (e.g., one or more devices of auxiliary system 108, etc.).

As shown in FIG. 9, at step 902, process 900 includes obtaining images. For example, management system 104 may obtain images. As an example, image capture system 102 may capture, over a period of time, a plurality of images of an environment (e.g., environment 100, etc.) surrounding the one or more image capture devices. In such an example, management system 104 may obtain the plurality of images from image capture system 102.

As shown in FIG. 9, at step 904, process 900 includes obtaining auxiliary data. For example, management system 104 may obtain auxiliary data. As an example, management system 104 may obtain, from a data source other than the one or more image capture devices (e.g., from auxiliary system 108, etc.), auxiliary data associated with at least one of: at least one image of the environment during the period of time and audio recorded in the environment during the period of time.

In some non-limiting embodiments or aspects, the medical device includes a catheter, and the auxiliary data includes at least one of: a tubing type of the catheter, a size of the catheter, a shape of the catheter, and a location of a catheter insertion site of the catheter on a patient.

In some non-limiting embodiments or aspects, the auxiliary data is associated with the audio recorded in the environment during the period of time. For example, the audio may include a predetermined signal associated with the medical device. As an example, the medical device may include an infusion pump, and the predetermined signal may include an audible signal emitted by the infusion pump (e.g., a power on sound, an indicator sound, etc.).

As shown in FIG. 9, at step 906, process 900 includes determining a location, a type, a trajectory, and/or an orientation of a medical device. For example, management system 104 may determine, based on the plurality of images and the auxiliary data, a location, a type, a trajectory, and/or an orientation of a medical device within the environment over the period of time. In some non-limiting embodiments or aspects, the medical device may not be visible (e.g., may not be detectable, etc.) in at least a portion of the plurality of images.

In some non-limiting embodiments or aspects, management system 104 may determine a plurality of locations of a plurality of medical devices 112 within the environment over the period of time and a plurality of types of the plurality of medical devices 112. For example, the plurality of medical devices 112 may include the medical device and the at least one other medical device, and the medical device and the at least one other medical device may be configured to emit a predetermined audible signal when connected (and/or disconnected). As an example, management system 104 may determine, based on the plurality of locations of the plurality of medical devices 112 within the environment over the period of time and the plurality of types of the plurality of medical devices 112, at least one connection (and/or disconnection) between two or more medical devices of the plurality of medical devices 112 and/or update, based on the at least one connection determined between the two or more medical devices, a database. In such an example, the audio included in the auxiliary data may include the predetermined audible signal emitted when the medical device and the at least one other medical are connected (and/or disconnected).

As shown in FIG. 9, at step 908, process 900 includes updating a database, providing an alert, and/or controlling one or more medical devices. For example, management system 104, based on the at least one determined event, may update a database including events associated with the environment, provide an alert associated with the at least one determined event, and/or control one or more medical devices in the environment.

In some non-limiting embodiments or aspects, in response to the at fluid delivery, management system 104 may at least one of: provide, to user device 106, an alert associated with the fluid delivery; and automatically control at least one medical device (e.g., a valve, an infusion pump, etc.) to stop a flow of fluid in a fluid flow path including the medical device associated with the at least one event.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A method comprising:
capturing, with one or more image capture devices, over a period of time, a plurality of images of an environment surrounding the one or more image capture devices;
determining, with one or more processors, based on the plurality of images, a plurality of locations of a plurality of medical devices within the environment over the period of time and a plurality of types of the plurality of medical devices;
determining, with the one or more processors, based on the plurality of locations of the plurality of medical devices within the environment over the period of time, a plurality of distances between the plurality of medical devices over the period of time;
determining, with the one or more processors, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and
updating, with the one or more processors, based on the at least one determined event, a database including current connections between the plurality of medical devices.

2. The method of claim 1, wherein the first medical device includes at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

3. The method of claim 1, further comprising:
determining, with the one or more processors, based on the plurality of distances between the plurality of medical devices over the period of time and the plurality of types of the plurality of medical devices, one of the following further events: (i) a reuse of the first medical device including a disconnection of the first medical device from the second medical device in the environment and a reconnection of the first medical device to the second medical device in the environment, (ii) a replacement of the first medical device with a new medical device of a same type as the first medical device including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and
updating, with the one or more processors, based on the further determined event, the database including the spatial relationships between the plurality of medical devices.

4. The method of claim 1, further comprising:
determining, with the one or more processors, based on the plurality of images, a plurality of orientations of the plurality of medical devices within the environment over the period of time, wherein determining the at least one event is further based on the plurality of orientations of the plurality of medical devices within the environment over the period of time.

5. The method of claim 4, wherein the database further includes directions of fluid flows in fluid flow paths through the current connections, wherein the at least one event includes the connection of the first medical device of the plurality of medical devices to the second medical device of the plurality of medical devices, and wherein the method further comprises:
determining, with the one or more processors, based on an orientation of the first medical device and an orientation of the second medical device, a direction of a fluid flow in a fluid plow path through the first medical device and the second medical device; and
updating, with the one or more processors, based on the direction of the fluid flow in the fluid plow path through the first medical device and the second medical device, the database.

6. The method of claim 1, wherein the first medical device includes at least one of a disinfectant cap and a disinfectant swab such that the connection of the first medical device of the plurality of medical devices to the second medical device of the plurality of medical devices does not form a fluid flow path through the first medical device and the second medical device.

7. The method of claim 1, wherein determining the at least one event further determines a probability associated with the at least one event.

8. The method of claim 1, further comprising:
delaying, with the one or more processors, determining the plurality of distances between the plurality of medical devices over the period of time and determining the at least one event until a location of at least one of the first medical device and the second medical device changes in the plurality of images over the period of time.

9. The method of claim 1, further comprising:
in response to determining the at least one event, at least one of:
providing, with the one or more processors, to a user device, an alert associated with the at least one event; and
automatically controlling, with the one or more processors, at least one medical device to stop a flow of fluid in a fluid flow path including the medical device.

10. The method of claim 1, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LIDAR sensor, or any combination thereof.

11. A system comprising:
one or more image capture devices configured to capture, over a period of time, a plurality of images of an environment surrounding the one or more image capture devices;
one or more processors programmed and/or configured to:
determine, based on the plurality of images, a plurality of locations of a plurality of medical devices within the environment over the period of time and a plurality of types of the plurality of medical devices;
determine, based on the plurality of locations of the plurality of medical devices within the environment over the period of time, a plurality of distances between the plurality of medical devices over the period of time;
determine, based on the plurality of distances between the plurality of medical devices over the period of time and the plurality of types of the plurality of medical devices, at least one event of the following events: (i) a connection of a first medical device of the plurality of medical devices to a second medical device of the plurality of medical devices and (ii) a disconnection of the first medical device of the plurality of medical devices from the second medical device of the plurality of medical devices; and
update, based on the at least one determined event, a database including current connections between the plurality of medical devices.

12. The system of claim 11, wherein the first medical device includes at least one of the following types of medical devices: a peripheral IV catheter (PIVC), a peripherally inserted central catheter (PICC), a midline catheter, a central venous catheter (CVC), a needleless connector, a catheter dressing, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

13. The system of claim 11, wherein the one or more processors are further programmed and/or configured to:
determine, based on the plurality of distances between the plurality of medical devices over the period of time and the plurality of types of the plurality of medical devices, one of the following further events: (i) a reuse of the first medical device including a disconnection of the first medical device from the second medical device in the environment and a reconnection of the first medical device to the second medical device in the environment, (ii) a replacement of the first medical device with a new medical device of a same type as the first medical device including the disconnection of the medical device from the at least one of the patient and the another medical device and a connection of the new medical device of the same type to the at least one of the patient and the another medical device, and (iii) a replacement of the medical device with different medical device of a different type than the medical device in the environment including the disconnection of the medical device from the t least one of the patient and the another medical device and a connection of the different medical device of the different type to the at least one of the patient and the another medical device; and
update, based on the further determined event, the database including the spatial relationships between the plurality of medical devices.

14. The system of claim 11, wherein the one or more processors are further programmed and/or configured to:
determine, based on the plurality of images, a plurality of orientations of the plurality of medical devices within the environment over the period of time, wherein determining the at least one event is further based on the plurality of orientations of the plurality of medical devices within the environment over the period of time.

15. The system of claim 14, wherein the database further includes directions of fluid flows in fluid flow paths through the current connections, wherein the at least one event includes the connection of the first medical device of the plurality of medical devices to the second medical device of the plurality of medical devices, and wherein the one or more processors are further programmed and/or configured to:
determine, based on an orientation of the first medical device and an orientation of the second medical device, a direction of a fluid flow in a fluid plow path through the first medical device and the second medical device; and
update, based on the direction of the fluid flow in the fluid plow path through the first medical device and the second medical device, the database.

16. The system of claim 11, wherein the first medical device includes at least one of a disinfectant cap and a disinfectant swab such that the connection of the first medical device of the plurality of medical devices to the second medical device of the plurality of medical devices does not form a fluid flow path through the first medical device and the second medical device.

17. The system of claim 11, wherein determining the at least one event further determines a probability associated with the at least one event.

18. The system of claim 11, wherein the one or more processors are further programmed and/or configured to:
delay determining the plurality of distances between the plurality of medical devices over the period of time and determining the at least one event until a location of at least one of the first medical device and the second medical device changes in the plurality of images over the period of time.

19. The system of claim 11, wherein the one or more processors are further programmed and/or configured to:
in response to determining the at least one event, at least one of:
provide, to a user device, an alert associated with the at least one event; and
automatically control at least one medical device to stop a flow of fluid in a fluid flow path including the medical device.

20. The system of claim 11, wherein the one or more image capture devices includes at least one of the following: a plurality of image capture devices, an infrared (IR) camera, a pan, tilt, and zoom (PTZ) camera including a variable field-of-view (FOV) and an automatic zoom function, a master and slave camera system including a static camera and a dynamic camera, a camera including a filter configured to filter a predetermined wavelength of light, a LiDAR sensor, or any combination thereof.
